(19) United States
(12) Patent Application Publication
(10) Pub. No.: US 2020/0069838 A1
(43) Pub. Date: Mar. 5, 2020

(54) PHOSPHATE FUNCTIONALIZED GRAPHENE OXIDE BASED BONE SCAFFOLDS

(71) Applicants: Carnegie Mellon University, Pittsburgh, PA (US); University of Connecticut, Farmington, CT (US)

(72) Inventors: Anne M. Arnold, Pittsburgh, PA (US); Brian D. Holt, Pittsburgh, PA (US); Stefanie A. Sydlik, Pittsburgh, PA (US); Cato T. Laurencin, Avon, CT (US); Leila Daneshmandi, Manchester, CT (US)

(21) Appl. No.: 16/610,814

(22) PCT Filed: May 3, 2018
(86) PCT No.: PCT/US2018/030967

§ 371 Date: Nov. 4, 2019

Related U.S. Application Data
(60) Provisional application No. 62/602,771, filed on May 5, 2017.

(51) Int. Cl. A61L 27/08; A61L 27/12; A61L 27/38; A61L 27/54; A61L 27/36; C01B 32/198

(52) U.S. Cl. CPC A61L 27/08; A61L 27/12; A61L 27/3834; A61F 2002/30985; A61L 27/365; C01B 32/198; A61L 2430/02; A61L 27/54

(57) ABSTRACT

A method for functionalizing graphene oxide includes reacting graphene oxide with a phosphite compound and a metal salt in the presence of a Lewis acid to produce phosphate functionalized graphene oxide including ions of the metal. An apparatus includes a bone scaffold construct formed of phosphate functionalized graphene oxide including metal ions. A bone scaffold construct includes a graphene oxide material formed in the shape of the bone scaffold construct, the graphene oxide material including graphene oxide, phosphate moieties covalently bound to the graphene oxide, and metal counter ions chemically associated with the phosphate moieties. A method for treating a bone defect includes administering a therapeutically effective amount of phosphate functionalized graphene oxide including metal ions.

### CLAIM OF PRIORITY

This application claims priority U.S. patent application Ser. No. 62/602,771, filed on May 5, 2017, the entire contents of which are incorporated here by reference.

### FEDERALLY SPONSORED RESEARCH OR DEVELOPMENT

This invention was made with government support under AR068147 awarded by the National Institutes of Health. The government has certain rights in the invention.

### BACKGROUND

Musculoskeletal injuries affect millions of patients worldwide on an annual basis. To treat musculoskeletal injuries, autografts (tissue grafts from the patient) or allografts (tissue grafts from a donor) can be used to treat severe injuries that either have delayed healing or cannot achieve union. Metal alloys can be employed as prosthetic devices for hard tissue regeneration.

### SUMMARY

In an aspect, a method for functionalizing graphene oxide includes reacting graphene oxide with a phosphite compound and a metal salt in the presence of a Lewis acid to produce phosphate functionalized graphene oxide including ions of the metal.

Embodiments can include one or more of the following features.

The metal salt includes a metal halide salt. The metal includes one or more of calcium, potassium, lithium, magnesium, and sodium. The ions of the metal include inducerons, such as inducerons capable of inducing osteogenesis or osteoinductivity. The phosphite compound includes an organophosphorous compound, such as triethylphosphite. The Lewis acid includes magnesium bromide diethyl etherate. Reacting the graphene oxide includes reacting the graphene oxide with the phosphite compound in a solution containing the Lewis acid; and adding the metal salt to the solution. Reacting the graphene oxide with the phosphite compound includes reacting epoxide moieties on the graphene oxide with the phosphite compound.

In an aspect, an apparatus includes a bone scaffold construct formed of phosphate functionalized graphene oxide including metal ions.

Embodiments can include one or more of the following features.

The bone scaffold construct is formed of a powder of the phosphate functionalized graphene oxide. The bone scaffold construct includes a putty. The bone scaffold construct comprises a membrane.

The bone scaffold construct has a compressive Young's modulus of greater than about 150 MPa, such as between about 150 MPa and about 3 GPa. An ultimate compressive strength of the bone scaffold construct is at least about 50 MPa, such as between about 50 MPa and about 350 MPa. Aa compressive storage modulus of the bone scaffold construct is at least about 100 MPa, such as between about 100 MPa and about 3GPa, such as between about 100 MPa and about 350 MPa. The compressive loss modulus of the bone scaffold construct is between about 5 MPa and about 3GPa, such as less than about 20 MPa, such as between about 5 MPa and about 20 MPa. A shear storage modulus of the bone scaffold construct is at least about 250 MPa, such as between about 250 MPa and about 3GPa, such as between about 250 MPa and about 650 MPa. A shear loss modulus of the bone scaffold construct is less than about 150 MPa, such as between about 40 MPa and about 3 GPa, such as between about 40 MPa and about 150 MPa. A toughness of the bone scaffold construct is between about 100 J·m-3·104 and about 3000 J·m-3·104.

The bone scaffold construct elutes metal ions when exposed to an aqueous environment. When the bone scaffold construct is exposed to an aqueous environment for a period of up to 28 days, a compressive modulus of the bone scaffold construct changes by less than about 100%, e.g., decreases by less than about 100%. When the bone scaffold construct is exposed to an aqueous environment for a period of up to 28 days, the compressive modulus of the bone scaffold construct changes by less than about 60%, such as less than about 40%, such as less than about 10%.

The metal ions include one or more of calcium ions, potassium ions, lithium ions, magnesium ions, and sodium ions. The metal ions include inducerons capable of inducing osteogenesis or osteoinductivity.

The bone scaffold construct includes an antimicrobial component, such as one or more of silver ions, copper ions, gallium ions, and zinc ions. The bone scaffold construct includes mesenchymal stem cells. The phosphate functionalized graphene oxide includes peptides covalently bound to the graphene oxide. The bone scaffold construct includes bioactive molecules non-covalently associated to the phosphate functionalized graphene oxide. The bioactive molecules include bone morphogenetic protein 2.

In an aspect, a bone scaffold construct includes a graphene oxide material formed in the shape of the bone scaffold construct, the graphene oxide material including graphene oxide, phosphate moieties covalently bound to the graphene oxide, and metal counter ions chemically associated with the phosphate moieties, the metal counter ions including one or more of calcium ions, potassium ions, lithium ions, magnesium ions, sodium ions, copper ions, manganese ions, strontium ions, vanadium ions, and zinc ions. A compressive Young's modulus of the graphene oxide material is between about 150 MPa and about 3 GPa. When the bone scaffold construct is exposed to an aqueous environment, the graphene oxide material elutes the metal counter ions.

Embodiments can have one or more of the following features.

The bone scaffold construct includes an antimicrobial component, such as one or more of silver ions, copper ions, gallium ions, and zinc ions. The bone scaffold construct includes mesenchymal stem cells. The graphene oxide material includes peptides covalently bound to the graphene oxide. The graphene oxide material includes bioactive molecules non-covalently associated to the graphene oxide. The bioactive molecules include bone morphogenetic protein 2.

In an aspect, a method for forming a bone scaffold construct includes forming a powder into the bone scaffold construct, the powder including phosphate functionalized graphene oxide including metal ions.

Embodiments can have one or more of the following features.

Forming the powder into the bone scaffold construct includes pressing the powder into the shape of the bone scaffold construct; and heat treating the pressed powder. Heat treating the pressed powder includes heat treating the pressed powder at 200° C. Forming the powder into the bone scaffold construct includes using an additive manufacturing technique to form the powder into the bone scaffold construct. Forming the powder into the bone scaffold construct comprises filtering a slurry of the powder to form a membrane. The metal ions include one or more of calcium ions, potassium ions, lithium ions, magnesium ions, and sodium ions. The metal ions include inducerons capable of inducing osteogenesis or osteoinductivity.

In an aspect, a method for forming a bone scaffold construct includes disposing a powder into a mold having the shape of the bone scaffold construct, the powder including phosphate functionalized graphene oxide including one or more of calcium ions, potassium ions, lithium ions, magnesium ions, and sodium ions; applying a compressive pressure to the powder in the mold to generate a pressed powder construct; removing the pressed powder construct from the mold; and heat treating the pressed powder at a temperature of between 175° C. and 225° C.

Embodiments can have one or more of the following features.

The method includes heating the mold and disposing the powder into the heated mold. Applying a compressive pressure to the powder in the mold includes applying a compressive pressure of at least about 1000 psi. The method includes sterilizing the bone scaffold construct.

In an aspect, a method for treating a bone defect includes administering a therapeutically effective amount of phosphate functionalized graphene oxide including metal ions.

Embodiments can have one or more of the following features.

The bone defect includes a birth defect, such as a cranial birth defect. The bone defect includes a bone fracture. The bone defect includes a loss of bone density due to osteoporosis. Administering the phosphate functionalized graphene oxide includes injecting an effective amount of a slurry of the phosphate functionalized graphene oxide into a site of the bone defect. Administering the phosphate functionalized graphene oxide includes surgically implanting a bone scaffold construct formed of the phosphate functionalized graphene oxide. The metal ions include one or more of calcium ions, potassium ions, lithium ions, magnesium ions, and sodium ions. The metal ions include inducerons capable of inducing osteogenesis. The method includes inducing osteogenesis on the phosphate functionalized graphene oxide. The presence of the metal ions induces osteogenesis. The method includes inducing osteoinductivity on the phosphate functionalized graphene oxide. The method includes eluting the metal ions from the phosphate functionalized graphene oxide.

### BRIEF DESCRIPTION OF DRAWINGS

FIGS. 1A and 1B are formulas of graphene oxide and phosphate-functionalized graphene oxide (X-PG), respectively.

FIG. 2 is an example scheme for synthesis of phosphate-functionalized graphene oxide.

FIG. 3 is a diagram of a patient with a bone defect.

FIG. 4 is a flow chart.

FIGS. 5A and 5B are Fourier Transform Infrared Spectroscopy (FTIR) plots for graphene oxide and X-PG.

FIG. 6 is a Thermogravimetric Analysis (TGA) plot for graphene oxide and X-PG.

FIGS. 7A and 7B are X-Ray Photoelectron Spectroscopy plots for graphene oxide and X-PG.

FIGS. 8A and 8B are FTIR plots for X-PG with calcium counter ions (CaPG).

FIG. 8C is a TGA plot for CaPG.

FIGS. 9A and 9B are plots of density and porosity, respectively, for graphene oxide and X-PG.

FIGS. 10A and 10B are graphs of storage moduli and loss moduli for graphene oxide and X-PG.

FIG. 11A is a plot of stress-strain curves for graphene oxide and X-PG.

FIG. 11B is a graph of toughness for graphene oxide and X-PG.

FIG. 11C is a graph of ultimate compressive strength for graphene oxide and X-PG.

FIG. 11D is a graph of Young's modulus for graphene oxide and X-PG.

FIGS. 12A and 12B are FTIR plots and TGA plots, respectively, for CaPG.

FIG. 13 is a plot of calcium concentration as a function of time.

FIG. 14 shows microscopy images of cells exposed to graphene oxide and X-PG.

FIGS. 15A and 15B show microscopy images of cells exposed to graphene oxide and X-PG.

FIGS. 16A and 16B are plots of alkaline phosphate expression and Alizarin Red S intensity, respectively.

FIGS. 16C and 16D are microscopy images of cells exposed to CaPG.

FIG. 17 shows gene expression in cells exposed to X-PG.

FIG. 18 shows microscopy images of cells exposed to X-PG.

FIGS. 19A and 19B are radiographs and quantifications of radiograph image intensity, respectively.

FIGS. 20A-20C are microscopy images of cells exposed to CaPG.

### DETAILED DESCRIPTION

We describe here a phosphate functionalized graphene oxide based material into which counter ions, such as calcium, potassium, lithium, magnesium, sodium ions, or other types of counter ions can be incorporated. Phosphate functionalized graphene oxide including counter ions can be used as a bone scaffold implant that has mechanical properties that mimic those of natural bone and that can induce the growth of bone cells. For instance, when implanted in a patient's body, the counter ions can be released from the phosphate functionalized graphene oxide and act as inducing factors (also called inducerons) to stimulate the differentiation of stem cells into osteoblasts. As a bone scaffold implant, phosphate functionalized graphene oxide can be used to treat bone defects, such as birth defects, bone fractures, bone deformities, bone density loss due to osteoporosis, or other types of bone defects.

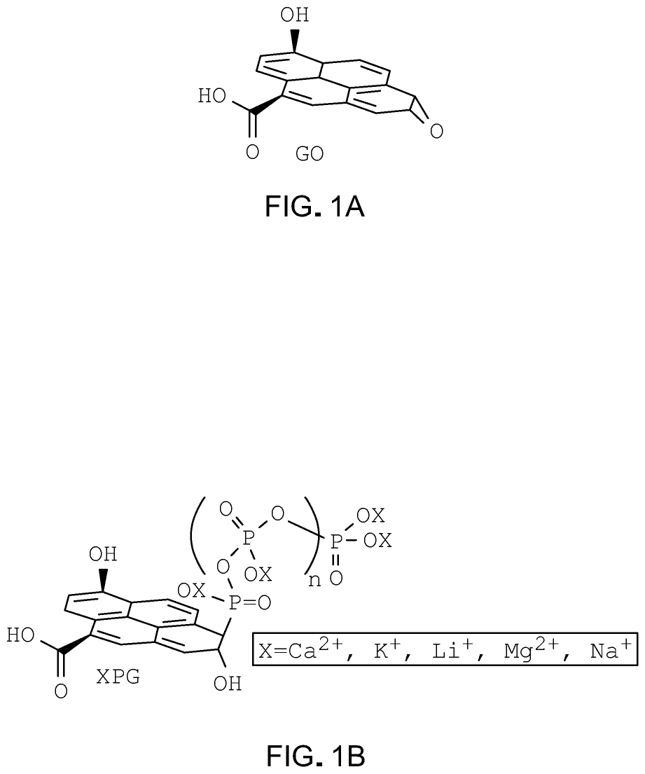

Referring to FIG. 1A, graphene oxide is a sheet-like, highly oxidized form of graphene that includes multiple oxygen-containing functionalities present on the edges and basal plane of its sheets. Graphene oxide is biocompatible in vitro and in vivo, and undergoes an autodegradation pathway in aqueous conditions, making graphene oxide potentially useful as a biodegradable medical device.

The oxygen-containing functionalities, such as hydroxyl groups, epoxide groups, carboxylic acid groups, or other types of oxygen-containing functionalities, provide avenues for chemical modifications of graphene oxide. For instance, referring to FIG. 1B, phosphate functionalities can be covalently incorporated into graphene oxide using epoxide groups on the basal plane of graphene oxide, forming phosphate-functionalized graphene oxide (sometimes referred to as PG).

The phosphate functionalities (referred to for simplicity as phosphates) can be associated with counter ions, denoted with an “X” in FIG. 1B. For instance, the counter ions can be ions that act as inducerons, which are signaling molecules for inductive tissue regeneration, such as inducerons that induce osteogenesis. Examples of osteogenic inducerons include metal ions such as calcium ions, potassium ions, lithium ions, magnesium ions, sodium ions, copper ions, manganese ions, strontium ions, vanadium ions, and zinc ions. Phosphate-functionalized graphene oxide with counter ions X is sometimes referred to as X-PG.

In some examples, X-PG can incorporate additional components, such as peptides covalently bound to the graphene oxide or bioactive molecules, e.g., bone morphogenetic protein 2 (BMP-2) non-covalently associated to the graphene oxide.

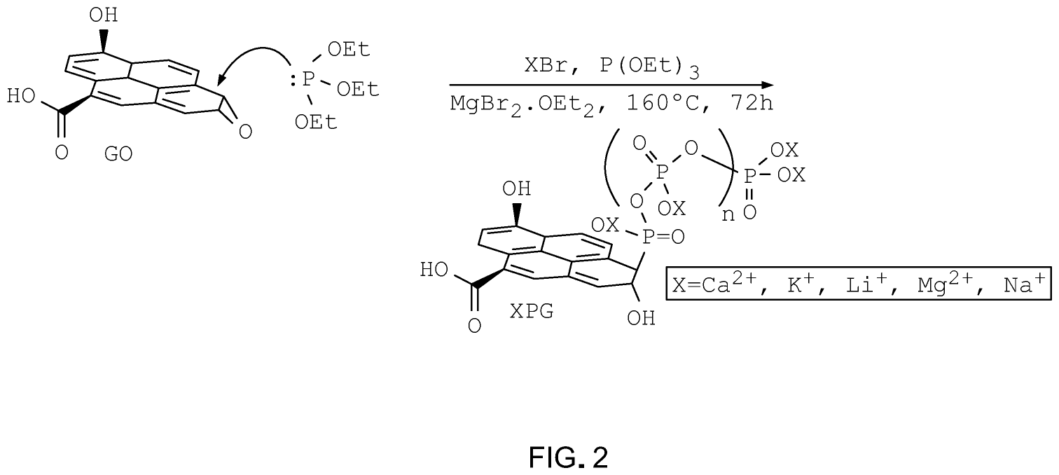

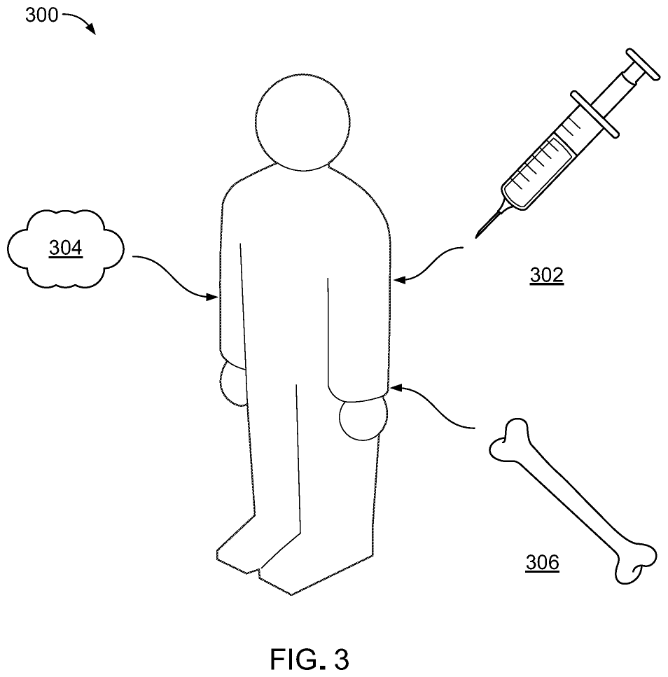

Referring to FIG. 2, to make phosphate-functionalized graphene oxide with metal counter ions, graphene oxide is reacted with a phosphite compound and a salt of the metal in the presence of a Lewis acid in a modified Arbuzov reaction. Without being bound by theory, it is believed that the Lewis acid acts as a catalyst, activating the epoxide moieties on the graphene oxide to facilitate phosphate functionalization. The identity of the metal counter ion can be controlled through selection of the metal salt.

The phosphite compound can be an organophosphorous compound, such as a trialkylphosphite, e.g., trimethylphosphite, triethylphosphite, or another organophosphorous compound. The mass ratio of phosphite compound to graphene oxide can be at least about 1:1, e.g., between about 1:1 and about 1000:1, such as between about 1:1 and about 500:1, between about 1:1 and about 100:1, or another range , e.g., about 1:1, about 2:1, about 10:1, about 50:1, about 100:1, about 500:1, about 1000:1, or another mass ratio.

The metal salt can be a metal halide salt, such as a metal bromide salt, e.g., calcium bromide, potassium bromide, lithium bromide, magnesium bromide, or sodium bromide. In some examples, other metal halide salts can be used, such as metal iodine salts, metal chloride salts, or other salts. The mole ratio of metal salt to graphene oxide in the reaction can be at least about 1:1, such as between about 1:1 to about 30:1, e.g., 1:1, 2:1, 5:1, 10:1, 15:1, 20:1, 25:1, 30:1, or another ratio. The mole ratio of metal salt to graphene oxide can depend on the identity of the metal. For instance, for metals that have low electropositivity, such as magnesium, a higher mole ratio can be used than for metals with higher electropositivity.

The Lewis acid can be a non-sterically hindered Lewis acid, such as a metal or a small compound. For instance, the Lewis acid can be Li⁺, Mg²+, BF₃, BCl₃, magnesium bromide diethyl etherate, or other Lewis acids. In some examples, the Lewis acid can be a non-bulky compound in which the empty orbital capable of accepting an electron pair from a donor species is not sterically hindered. The mass ratio of Lewis acid to graphene oxide can be at least about 1:100, e.g., between about 1:100 and about 100:1, such as between about 1:100 and about 1:1, between about 1:10 and about 1:1, between about 1:10 and about 10:1, between about 10:1 and about 1:1, between about 100:1 and about 1:1, or another range, e.g., about 1:100, about 1:50, about 1:10, about 1:1, about 10:1, about 50:1, about 100:1, or another mass ratio.

In an example process for synthesizing X-PG, graphene oxide is reacted with the phosphite compound in the presence of the Lewis acid to produce phosphate-functionalized graphene oxide (PG). For instance, a mixture of graphene oxide, the phosphite compound, and the Lewis acid can be stirred or sonicated under an inert atmosphere, e.g., under nitrogen, argon, or another inert atmosphere. The mixture can be stirred or sonicated for at least about 15 minutes, e.g., about 15 minutes, about 30 minutes, about 1 hour, about 2 hours, or another amount of time. The mixture can be stirred or sonicated at a temperature of between about 15° C. and about 60° C., such as between about 15° C. and about 30° C., between about 30° C. and about 60° C., between about 30° C. and about 45° C., or another range, e.g., about 15° C., about 30° C., about 45° C., about 60° C., or another temperature. The metal salt is then added to the mixture and stirred or sonicated for at least about 15 minutes, e.g., about 15 minutes, about 30 minutes, about 1 hour, about 2 hours, or another amount of time. The mixture can be stirred or sonicated at a temperature of between about 15° C. and about 60° C., such as between about 15° C. and about 30° C., between about 30° C. and about 60° C., between about 30° C. and about 45° C., or another range, e.g., about 15° C., about 30° C., about 45° C., about 60° C., or another temperature.

Following the stirring or sonication, the reaction is refluxed at elevated temperature, such as between about 150° C. and about 200° C., e.g., 150° C., 160° C., 180° C., or 200° C., under an inert atmosphere. The refluxing can be carried out for at least 12 hours, e.g., 12 hours, 24 hours, 48 hours, 72 hours, 96 hours, or another amount of time. The resulting X-PG material can be recovered by filtration, centrifugation, or washing.

Referring to FIG. 3, X-PG materials can be used as bone scaffold constructs. For instance, X-PG materials can be injected or implanted into a patient's body 300 for treatment of a bone defect, such as a birth defect, such as a cranial birth defect; a bone fracture; a loss of bone density due to osteoporosis; or another type of bone defect. X-PG materials can be injected as a dispersion of X-PG material in a liquid 302, implanted as a viscous putty 304, or implanted as a solid, three-dimensional construct 304 of X-PG material. The presence of X-PG material, such as CaPG or other X-PG materials, in a cellular environment can inspire osteogenesis, as discussed further below. The injection or implantation of X-PG materials into a patient's body in a target area can thus inspire osteogenesis or osteoinductivity in that area, facilitating treatment of the patient's bone defect. Osteogenesis is the regeneration of bone cells. Osteoinductivity is the perpetuation of bone cells and phenotype.

In some examples, bone scaffold constructs formed of X-PG materials can include antimicrobial agents, such as metal ions having antimicrobial properties, e.g., silver ions, copper ions, gallium ions, zinc ions, or other antimicrobial ions. In some examples, bone scaffold constructs formed of X-PG materials can include stem cells, such as mesenchymal stem cells. In some examples, the X-PG material of a bone scaffold construct can include peptides covalently bound to the graphene oxide, e.g., to promote cell adhesion to the bone scaffold construct, to stimulate osteogenesis or osteoinductivity, or for other purposes. In some examples, the X-PG material of a bone scaffold construct can include bioactive small molecules, e.g., bone morphogenetic protein 2 (BMP-2), non-covalently associated with the graphene oxide.

In some examples, dispersions of X-PG materials in a liquid, such as in water or in a buffer solution, can be injected into a patient in a target area, such as an area having a bone defect. The presence of X-PG material in the patient's body inspires the growth of new bone cells, thus facilitating healing of the bone defect. In some examples, a therapeutically effective amount of X-PG dispersion can be based on a weight of the patient, a size of the target area, an extent of the bone defect, or another factor. For instance, when the therapeutically effective amount is based on patient weight, the amount can be between about 10 mg/kg and about 500 mg/kg, such as between about 10 mg/kg and about 50 mg/kg, between about 10 mg/kg and about 100 mg/kg, between about 100 mg/kg and about 500 mg/kg, or another range, e.g., about 10 mg/kg, about 20 mg/kg, about 50 mg/kg, about 100 mg/kg, about 200 mg/kg, about 300 mg/kg, about 400 mg/kg, about 500 mg/kg, or another amount.

In some examples, X-PG materials can be formed into a viscous putty that can be molded, e.g., by hand or using a molding tool, into a desired shape. The putty can be molded prior to implantation in a patient or during the implantation. For instance, the putty can be molded during implantation to fit a specific shape or configuration of a bone defect under treatment. In some examples, a therapeutically effective amount of X-PG material sufficient to facilitate treatment of a bone defect can be a volume of putty that is sufficient to fill or coat all or a portion of a region of a bone defect. For instance, a viscous putty of X-PG material can be pressed into a crack in a patient's bone, partially or completely filling the bone and inspiring osteogenesis or osteoinductivity in the region of the crack.

X-PG materials can be formed into solid constructs that can be used as bone scaffolds. For instance, solid X-PG constructs can be formed in shapes that can be joined with existing bone or other tissue in a patient's body. In general, solid X-PG constructs can be fabricated using approaches that do not substantially degrade the covalent phosphate functionalization of the graphene oxide. For instance, solid X-PG constructs can be fabricated from X-PG powder as a starting material using heat treatments that do not exceed a critical temperature of the X-PG powders. For instance, the critical temperature of the X-PG powders can be between about 200° C. and about 300° C., e.g., between about 240° C. and about 270° C., e.g., between about 246° C. and about 266° C.

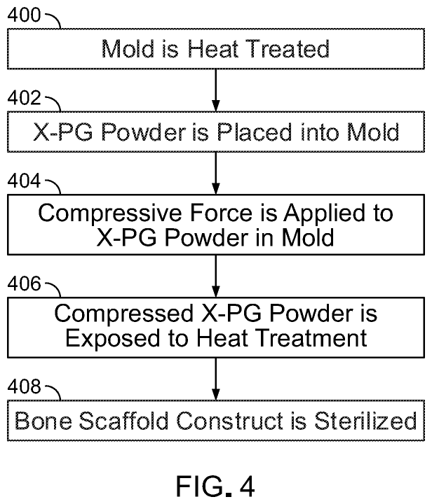

In some examples, solid X-PG constructs can be fabricated by compressing and heat treating X-PG powders. For instance, referring to FIG. 4, in an example process for fabricating a solid X-PG construct, a mold having a target shape, such as the shape of a bone scaffold to be used to inspire osteogenesis or osteoinductivity in a patient, is heat treated (400). For instance, the mold is heated in an oven to a temperature of between about 150° C. and about 250° C., e.g., 150° C., 175° C., 200° C., 225° C., or 250° C. The mold is removed from the oven and X-PG powder is placed into the mold (402). A compressive force is applied to the powder to compress the powder into a solid (404). For instance, the compressive force is applied for up to 5 minutes, e.g., 1 minute, 2 minutes, 5 minutes, or another amount of time. The compressive force can be at least about 1000 psi, e.g., between about 1000 psi and about 10,000 psi. The solid is removed from the mold and exposed to a heat treatment (406). For instance, the solid is heat treated at a temperature between about 150° C. and about 250° C., e.g., 150° C., 175° C., 200° C., 225° C., or 250° C. The heat treatment can be carried out for at least 15 minutes, e.g., between 15 minutes and 2 hours, e.g., 15 minutes, 20 minutes, 30 minutes, 1 hour, 2 hours, or another amount of time. Prior to implantation of the bone scaffold construct into a patient, the bone scaffold construct is sterilized (408), e.g., by gamma radiation.

In some examples, solid X-PG constructs can be fabricated from X-PG powders using an additive manufacturing technique, such as 3D printing, rapid prototyping, or other types of additive manufacturing. In additive manufacturing techniques, the shape of the solid X-PG construct can be tailored to a target application through digital control of the additive manufacturing process. For instance, a solid X-PG construct for use as a bone scaffold implant can be fabricated with customized shape and dimensions for use with a specific patient.

In some examples, solid X-PG constructs can be membranes fabricated by filtering a slurry of X-PG powder in a liquid, such as water. For instance, a slurry of X-PG powder in liquid can be filtered through a filtration device, such as filter paper or a frit, e.g., by vacuum filtration. The filtration removes the water from the slurry, leaving a membrane of X-PG material disposed on the filtration device. The membrane can be a free-standing membrane that can be removed from the filtration device. The slurry can have a ratio of X-PG material to water of between about 1:1 and about 1:100, such as between about 1:1 and about 1:10, between about 1:1 and about 1:50, between about 1:10 and about 1:100, between about 1:50 and about 1:100, or another ratio. For instance, the slurry can have a ratio of X-PG material to water of about 1:1, about 1:10, about 1:20, about 1:50, about 1:100, or another ratio.

Solid X-PG constructs can have mechanical properties, such as compressive and shear moduli, compressive strength, and toughness, that are generally on the order of the mechanical properties of hard tissue, such as native bone tissue. The compressive mechanical properties of X-PG materials can be independent of the strain rate applied to the materials. For instance, when used as a bone scaffold implant, solid X-PG constructs have mechanical properties enabling the constructs to withstand loads associated with physical activities, such as walking and running, without compromising the mechanical integrity of the material.

Solid X-PG constructs can have a bulk density less than the bulk density of graphene oxide, e.g., between about 1.4 and about 1.8 g/cm³. The porosity of solid X-PG constructs can be higher than the porosity of graphene oxide, e.g., at least about 20% porosity, e.g., between about 20% and about 40% porosity. Without being bound by theory, it is believed that the lower density and higher porosity of solid X-PG constructs may be due to phosphate functionalization, which increases the interlayer distance between sheets of graphene oxide. The density and porosity of solid X-PG constructs can depend on the identity of the counter ion. In a specific example, the density of CaPG is between about 1.7 and 1.9 g/cm³, e.g., 1.77 g/cm³; and the porosity of CaPG is between 20% and 22%, e.g., 21.4%.

The compressive Young's modulus (E) of a solid X-PG construct can be at least about 150 MPa, such as between about 150 MPa and about 3 GPa, between about 150 MPa and about 2 GPa, between about 150 MPa and about 1 GPa, between about 1 GPa and about 3 GPa, between about 1.5 GPa and about 3 GPa, between about 1 GPa and about 2 GPa, between about 2 GPa and about 3 GPa, or another range. For instance, the compressive Young's modulus of a solid X-PG construct can be about 150 MPa, about 500 MPa, about 1 GPa, about 1.5 GPa, about 2 GPa, about 2.5 GPa, about 3 GPa, or another value. The Young's modulus can depend on the identity of the counter ion X. For instance, CaPG, KPG, and NaPG can have a Young's modulus between about 1.5 GPa and about 2.0 GPa. LiPG and MgPG can have a Young's modulus of between about 1.0 GPa and about 1.3 GPa. In a specific example, the Young's modulus of CaPG can be about 1.8 GPa.

The compressive storage modulus (E′) of a solid X-PG construct can be at least 100 MPa, such as between about 100 MPa and about 3 GPa, between about 100 MPa and about 2 GPa, between about 100 MPa and about 1 GPa, between about 100 MPa and about 500 MPa, between about 100 MPa and about 350 MPa, between about 1 GPa and about 3 GPa, or another range. For instance, the compressive storage modulus of a solid X-PG construct can be about 100 MPa, about 200 MPa, about 300 MPa, about 350 MPa, about 500 MPa, about 1 GPa, about 1.5 GPa, about 2 GPa, about 2.5 GPa, about 3 GPa, or another value. The compressive storage modulus can depend on the identity of the counter ion X. For instance, the compressive storage modulus of CaPG can be between about 180 MPa and about 200 MPa, e.g., about 180 MPa, about 190 MPa, or about 200 MPa. The compressive storage modulus of KPG and LiPG can be between about 150 MPa and about 170 MPa, e.g., about 150 MPa, about 160 MPa, or about 170 MPa. The compressive storage modulus of MgPG can be between about 200 MPa and about 220 MPa, e.g., about 200 MPa, about 210 MPa, or about 220 MPa. The compressive storage modulus of NaPG can be between about 280 MPa and about 300 MPa, e.g., about 280 MPa, about 290 MPa, or about 300 MPa. These values for the compressive storage modulus are on the order of the compressive storage modulus for hard tissue, such as trabecular bone.

The compressive loss modulus (E″) of a solid X-PG construct can be less than about 3 GPa, such as less than 2 GPa, less than 1 GPa, less than 500 MPa, less than 100 MPa, less than 20 MPa, e.g., between about 5 MPa and about 3 GPa, between about 5 MPa and about 2 GPa, between about 5 MPa and about 1 GPa, between about 5 MPa and about 500 MPa, between about 5 MPa and about 100 MPa, between about 5 MPa and about 20 MPa, or another range.

The shear storage modulus (G′) of a solid X-PG construct can be at least 250 MPA, such as between about 250 MPa and about 3 GPa, between about 250 MPa and about 2 GPa, between about 250 MPa and about 1 GPa, between about 250 MPa and about 650 MPa, between about 1 GPa and about 3 GPa, or another range. For instance, the compressive storage modulus of a solid X-PG construct can be about 250 MPa, about 300 MPa, about 400 MPa, about 500 MPa, about 600 MPa, about 650 MPa, about 1 GPa, about 1.5 GPa, about 2 GPa, about 2.5 GPa, about 3 GPa, or another value. The shear storage modulus can depend on the identity of the counter ion X. For instance, the shear storage modulus of CaPG can be between about 530 MPa and about 550 MPa, e.g., about 530 MPa, about 540 MPa, or about 550 MPa. The shear storage modulus of KPG can be between about 510 MPa and about 530 MPa, e.g., about 510 MPa, about 520 MPa, or about 530 MPa. The shear storage modulus of LiPG can be between about 290 MPa and about 310 MPa, e.g., about 290 MPa, about 300 MPa, or about 310 MPa. The shear storage modulus of MaPG can be between about 350 MPa and about 370 MPa, e.g., about 350 MPa, about 360 MPa, or about 370 MPa. The shear storage modulus of NaPG can be between about 510 MPa and about 530 MPa, e.g., about 510 MPa, about 520 MPa, or about 530 MPa.

The shear loss modulus (G″) of a solid X-PG construct can be less than about 3 GPa, such as less than 2 GPa, less than 1 GPa, less than 500 MPa, less than 150 MPa, e.g., between about 40 MPa and about 3 GPa, between about 40 MPa and about 2 GPa, between about 40 MPa and about 1 GPa, between about 40 MPa and about 500 MPa, between about 40 MPa and about 150 MPa.

The ultimate compressive strength of a solid X-PG construct can be between about 50 MPa and about 350 MPa, such as between about 50 MPa and about 200 MPa, between about 50 MPa and about 100 MPa, between about 100 MPa and about 350 MPa, between about 200 MPa and about 350 MPa, between about 100 MPa and about 200 MPa, or another range. For instance, the ultimate compressive strength of a solid X-PG construct can be about 50 MPa, about 100 MPa, about 150 MPa, about 200 MPa, about 250 MPa, about 300 MPa, about 350 MPa, or another value. The ultimate compressive strength can depend on the identity of the counter ion X. For instance, CaPG can have an ultimate compressive strength between about 250 MPa and about 300 MPa. KPG can have an ultimate compressive strength between about 200 MPa and about 250 MPa. MgPG can have an ultimate compressive strength between about 150 MPa and about 200 MPa. NaPG can have an ultimate compressive strength between about 300 MPa and about 350 MPa. In a specific example, the ultimate compressive strength of CaPG can be about 254 MPa. As a comparison, the ultimate compressive strength of graphene oxide is between about 150 MPa and about 200 MPa.

The toughness of a solid X-PG construct can be between about 100 J·m⁻³·4 and about 3000 J·m⁻³·10⁴, e.g., about 100 J·m⁻³·10⁴, about 500 J·m⁻³·10⁴, about 1000 J·m⁻³·10⁴, about 1500 J·m⁻³·10⁴, about 2000 J·m⁻³·10⁴, about 2500 J·m⁻³·10⁴, about 3000 J·m⁻³·10⁴, or another value. The toughness can depend on the identity of the counter ion X. For instance, the toughness of CaPG can be between about 1800 J·m⁻³·10⁴ and about 1900 J·m⁻³·10⁴. The toughness of KPG can be between about 1800 J·m⁻³·10⁴ and about 1900 J·m⁻³·10⁴. The toughness of MgPG can be between about 1500 J·m⁻³·10⁴ and about 1600 J·m⁻³·10⁴. The toughness of NaPG can be between about 2300 J·m⁻³·10⁴ and about 2400 J·m⁻³·10⁴. In a specific example, the toughness of CaPG can be about 1817 J·m⁻³·10⁴. As a comparison, the toughness of graphene oxide is between about 1500 J·m⁻³·10⁴ and about 1600 J·m⁻³·10⁴.

Table 1 lists example average mechanical properties and ranges of mechanical properties for solid X-PG constructs. The Range values in Table 1 indicate the mean and standard deviations of the CaPG, KPG, LiPG, MgPG, and NaPG materials. Average_{Low} is the Range_{Low} value minus the standard deviation and Average_{High} is the Range_{High} value plus the standard deviation.

TABLE 1
Example mechanical properties of solid X-PG constructs.

| | Average_{Low} | Average_{High} | Range_{Low} | Range_{High} |
|---|---|---|---|---|
| Property | (MPa) | (MPa) | (MPa) | (MPa) |
| Compressive | 1100 | 1800 | 750 | 2500 |
| Young's Modulus/ | | | | |
| Stiffness (E) | | | | |
| Ultimate | 57 | 300 | 50 | 350 |
| Compressive | | | | |
| Strength (UCS) | | | | |
| Compressive | 159 | 291 | 100 | 325 |
| Dynamic | | | | |
| Mechanical | | | | |
| Analysis | | | | |
| Storage Modulus | | | | |
| (E′) | | | | |
| Compressive | 10 | 15 | 8 | 18 |
| Dynamic | | | | |
| Mechanical | | | | |
| Analysis Loss | | | | |
| Modulus (E″) | | | | |
| Viscoelastic | 295 | 543 | 250 | 650 |
| Torsional | | | | |
| Shear Storage | | | | |
| Modulus (G′) | | | | |
| Viscoelastic | 61 | 122 | 40 | 150 |
| Torsional | | | | |
| Shear Loss | | | | |
| Modulus (G″) | | | | |
| Toughness (U_{T}) | 156 | 2326 | 100 | 3000 |

The compressive mechanical properties of solid X-PG constructs can remain substantially stable in an aqueous environment, e.g., enabling X-PG materials to be used as long-term, mechanically stable bone scaffold implants. Specifically, when used as a bone scaffold, solid X-PG constructs are exposed to an aqueous environment in a patient's body, and mechanical stability of the X-PG material in an aqueous environment can contribute to preservation of the structural integrity of the bone scaffold. In some examples, solid X-PG constructs are stable in an ex vivo aqueous environment for several days, such as at least 5 days, at least 10 days, at least 15 days, at least 20 days, at least 25 days, at least 28 days, or at least 30 days, or longer. A solid X-PG construct is considered to be stable in an aqueous environment over a period of time if a mechanical modulus, such as the Young's modulus or a compressive modulus (e.g., a compressive storage modulus or a compressive loss modulus) of the construct changes (e.g., increases or decreases) less than about 100% over the period of time, e.g. less than 80%, less than 60%, less than 50%, less than 40%, less than 20%, less than 10%, or another amount over the period of time.

In some examples, X-PG materials, such as solid X-PG constructs, elute counter ions into solution when exposed to an aqueous environment. When used as a bone scaffold implant, the elution of counter ion inducerons, such as calcium ions, can induce osteogenesis or osteoinductivity in the vicinity of the bone scaffold implant, thus facilitating bone growth and enabling the bone scaffold implant to be used for tissue engineering applications. As the elution of counter ions proceeds, the X-PG material can degrade to phosphate-functionalized graphene or to graphene oxide, both of which are stable and tolerated in in vitro and in vivo environments.

The elution of counter ions from X-PG materials into solution can be quantified using an ocresolphthalein complexone chelator colorimetric assay. In an example, solid CaPG constructs in a phosphate buffered saline (PBS) solution elute calcium ions, e.g., up to about 500 μM per mg of CaPG, such as about 100 μM per mg, about 200 μM per mg, about 300 μM per mg, about 400 μM per mg, about 500 μM per mg, or another amount. The elution of calcium ions can stabilize after at least about 5 days in solution, e.g., about 5 days, about 10 days, about 15 days, about 20 days, or another amount of time. Elution of ions is considered to be stabilized at a point in time when the change in ion concentration in solution is less than about 10%, e.g., less than about 5%, or less than about 2%, after that point in time.

The elution of counter ions, such as calcium ion inducerons, from X-PG materials can be a diffusion controlled process. For instance, X-PG materials can be used in tissue engineering applications, e.g., osteogenic or osteoinductive applications, for diffusion-controlled delivery of therapeutic bioactive moieties, such as osteogenic induceron ions. As the elution of osteogenic inducerons proceeds, inspiring the growth of bone cells, the material can degrade to phosphate-functionalized graphene or graphene oxide, which can act as a stable, mechanically robust scaffold for the growing bone tissue.

X-PG materials can have properties, such as particle size and Zeta potential, that are generally sufficient for compatibility with cells (referred to as cytocompatibility), such as animal cells, e.g., fibroblasts, macrophages, osteoblasts, or other types of cells. For instance, the particle size of a dispersion of X-PG material can be in a range that is sufficient for cytocompatibility, e.g., a dispersion of X-PG material in an aqueous environment can have a particle size of between about 2 μm and about 20 μm, e.g., about 2 μm, about 5 μm, about 10 μm, about 15 μm, or about 20 μm. The Zeta potential of X-PG material can be in a range that is sufficient for cytocompatibility. For instance, the Zeta potential of a dispersion of X-PG material in water at a concentration of 100 μg/mL can be between about −20 mV and about −60 mV. Furthermore, X-PG materials can be compatible with cellular vitality, e.g., cell proliferation and metabolism, and can have little deleterious effect on sub-cellular compartments, e.g., nuclei, filamentous actin, or mitochondria, of cells exposed to the X-PG materials.

In some examples, X-PG materials can facilitate cellular growth, such as growth of fibroblasts (e.g., NIH-3T3 fibroblasts) or human mesenchymal stem cells (hMSCs). The rate of cell proliferation facilitated by X-PG materials can depend on the identity of the X-PG counter ion. Without being bound by theory, it is believed that this difference may be due to one or more of the potency of each counter ion as an induceron, the release rate of the counter ion from the X-PG material, and the interfacial topology of the X-PG material.

In some examples, X-PG materials can induce differentiation of stem cells, such as mesenchymal stem cells, into an osteoblastic phenotype. X-PG materials that can induce stem cell differentiation can be used for tissue engineering applications. For instance, X-PG materials, such as CaPG can be used as bone scaffold implants for tissue engineering applications. These materials can promote osteogenic differentiation through release of inducerons, such as calcium ions. Furthermore, the mechanical properties of solid X-PG materials provides stiffness and mechanical integrity that enable the X-PG material to act as a substantive scaffold during osteogenesis or osteoinductivity.

In some examples, exposure of hMSCs to X-PG materials can result in differentiation of hMSCs toward osteoblastic phenotype, indicating the ability of X-PG materials to inspire osteogenesis or osteoinductivity. For instance, hMSC differentiation can be measured by evaluation of the expression of alkaline phosphatase (ALP), which is highly expressed in osteoblasts. In an example, for hMSCs exposed to CaPG, the ALP expression can increase by at least about 100% over a period of 10 days, such as between about 100% and about 400%, e.g., about 100%, about 200%, about 300%, about 400%, or another amount, indicating the increasing differentiation of hMSCs toward osteoblastic phenotype. hMSC differentiation can also be measured by evaluation of the intensity of Alizarin Red S (ARS), which labels calcium deposits that are indicative of mineralization from cells displaying an osteogenic phenotype. In an example, for hMSCs exposed to CaPG, the ALS intensity can increase by at least about 100% over a period of 28 days, such as between about 100% and about 200%, e.g., about 100%, about 150%, about 200%, or another amount. In some examples, exposure of hMSCs to CaPG materials results in an increased level of expression of osteogenic genes of hMSCs, such as collagen type I alpha 1 (COL1A1), bone morphogenetic protein 2 (BMP-2), and runt-related transcription factor 2 (RUNX-2) as measured by PCR.

Without being bound by theory, it is believed that CaPG mimics natural bony apatite, and solid CaPG constructs controllably release calcium ion inducerons in a diffusion-controlled release process that can stimulate hMSC differentiation.

### EXAMPLES

The following examples demonstrate the synthesis and characterization of phosphate functionalized graphene oxide (X-PG) and the fabrication and chemical and mechanical characterization of solid X-PG constructs. The examples also demonstrate the cytocompatibility of X-PG materials and the ability of X-PG materials to induce osteogenesis or osteoinductivity.

### Example 1—Synthesis of Phosphate Functionalized Graphene Oxide

Graphene oxide (GO) was synthesized from graphite using a modified Hummers' method. The reaction was run using 10 g of graphite flakes (graphite flake, natural, −325 mesh, 99.8% metal basis; Alfa Aesar, Ward Hill, Mass., USA) that was added to a 1 L flask containing 250 mL of concentrated sulfuric acid (Fisher Scientific, Pittsburgh, Pa., USA) cooled over ice while stirring. Then, 20 g of KMnO₄ (Sigma-Aldrich, St. Louis, Mo., USA) was slowly added over 20-30 min. The reaction was warmed to room temperature and stirred for 2 h followed by gentle heating to 35° C. and stirring for an additional 2 h. The heat was then removed and the reaction was quenched by slowly adding 1400 mL of deionized (DI) water followed by the slow addition of 20 mL of 30% H₂O₂ (Fisher Scientific). Lastly, 450 mL of DI water was added, and the reaction stirred overnight.

To purify the graphene oxide, the reaction mixture was centrifuged at 3,600×g for 5 min. The resulting pellet was collected and loaded into 3,500 molecular weight cutoff dialysis tubing (SnakeSkin™ dialysis tubing; Thermo Scientific, Waltham, Mass., USA) and dialyzed against DI water for 3-7 days. The DI water was changed 2 times the first day and then once a day until the water was clear. Following dialysis, the graphene oxide was frozen at −80° C. and lyophilized for 3-5 days to dryness.

Phosphate modified graphene oxide was prepared from graphene oxide in a modified Arbuzov reaction using a Lewis acid (magnesium bromide diethyl etherate) to facilitate the reaction. 500 mg of graphene oxide, 500 mL of triethyl phosphite (Sigma Aldrich), and 500 mg of magnesium bromide diethyl etherate (Alfa Aesar) were loaded into a flame dried round bottom flask under Nz. The reaction mixture was sonicated (240 W, 42 kHz, ultrasonic cleaner, Kendal) for 1 h followed by the addition of the appropriate anhydride metal bromide salt: 2.5 g of calcium bromide (Alfa Aesar), 2.5 g of potassium bromide (Alfa Aesar), 2.5 g of lithium bromide (Oakwood Chemicals, Estill, S.C., USA), 12.5 g of magnesium bromide (Alfa Aesar), or 2.5 g of sodium bromide (Alfa Aesar). The reaction was sonicated for an additional 30 min. The reaction was refluxed at 160° C. under N2 with stirring for 72 h.

The phosphate modified graphene oxide materials were purified by vacuum filtering the reaction and collecting the filter puck and discarding the filtrate. The resulting product was washed with acetone and centrifuged at 3,600×g for 5 min. The supernatant was discarded, and the pellet was re-dispersed in fresh solvent for additional wash steps. The pellet was washed once more with acetone, once with ethanol, once with DI water, and an additional two washes with acetone. The resulting pellet was dried under vacuum for 24-48 h until dry.

### Example 2—Chemical Characterization of Phosphate Modified Graphene Oxide

Phosphate modified graphene oxides were characterized to determine the extent and effectiveness of the phosphate modification of graphene oxide. Specifically, graphene oxide and X-PG powders containing Ca, K, Li, Mg, or Na as the counter ion X (referred to as CaPG, KPG, LiPG, MgPG, and NaPG, respectively) were characterized by Fourier Transform Infrared Spectroscopy (FTIR), Thermogravimetric Analysis (TGA), and X-Ray Photoelectron Spectroscopy (XPS).

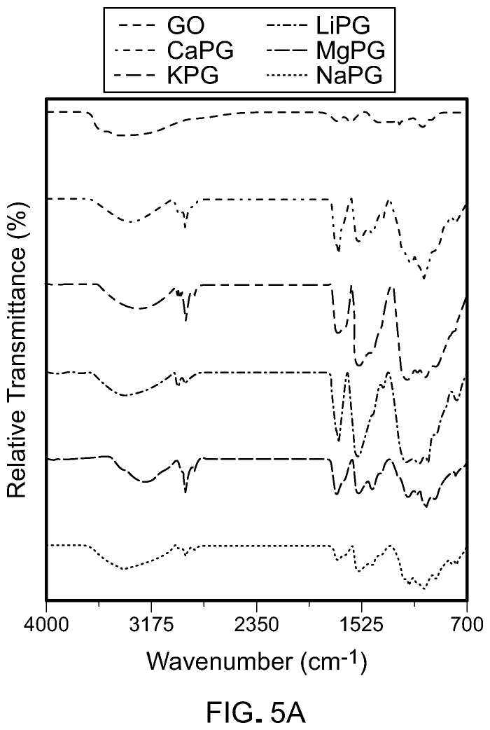

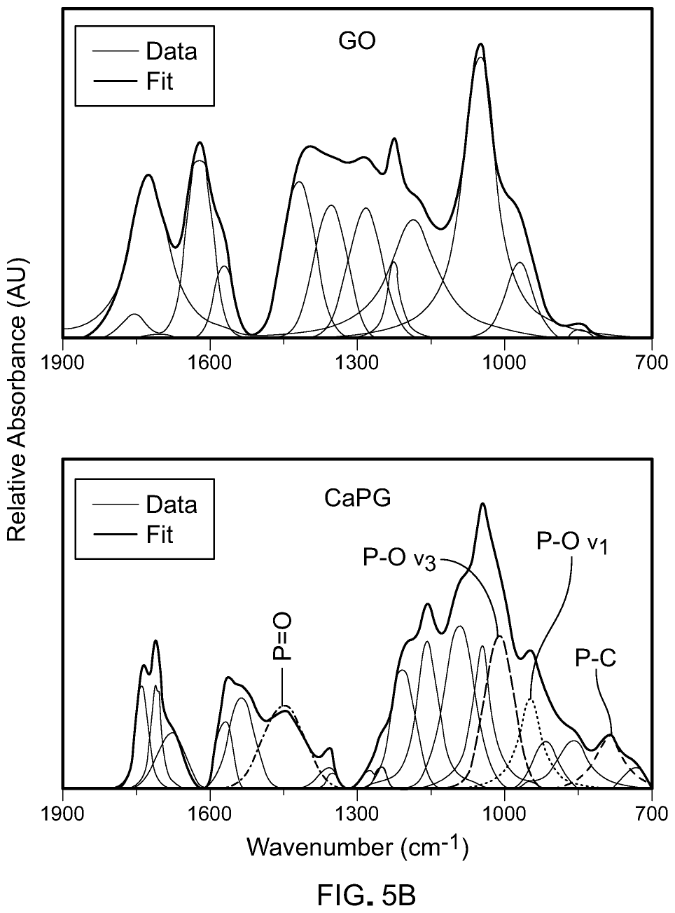

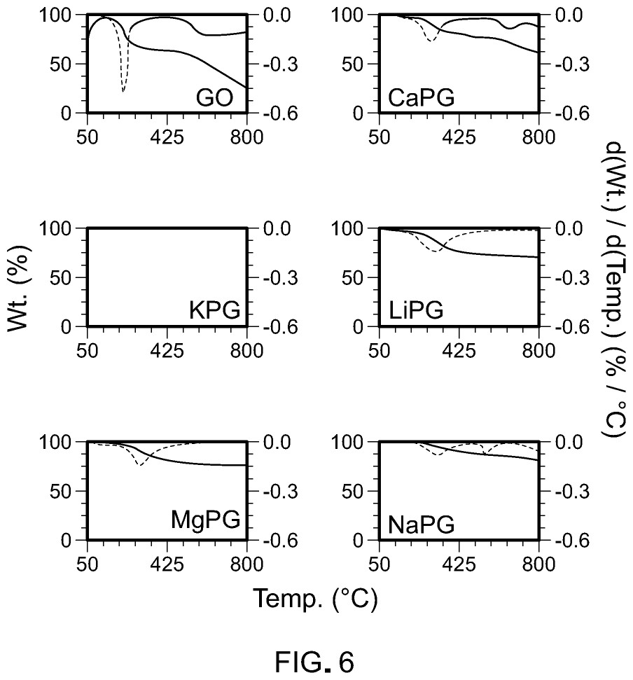

Referring to FIG. 5A, FTIR characterization of graphene oxide and X-PG revealed strong FTIR stretches in the 1000-1200 cm⁻¹ range, shown in the shaded region of FIG. 1A. These FTIR stretches are indicative of phosphate functionalization of the graphene oxide sheets. Referring to FIG. 5B, a peak deconvolution of FTIR fingerprint regions showed the existence of phosphate peaks that are unique to phosphate-functionalized graphene oxide materials. The presence of the P—C peak is indicative of covalent phosphate functionalization on the graphene backbone.

Referring to FIG. 5, TGA of graphene oxide shows a well-defined degradation event for the evolution of labile oxygen groups. For X-PG materials, there was a clear increase in the onset temperature (To) and first derivative peak temperature (Tₚ) of the degradation event. These shifts of 55-75° C. and 81-89° C., respectively, are indicative of phosphorus-carbon bond installations that evolve at higher temperatures, marking the presence of phosphate functionalization. In FIG. 5, the solid lines are the TGA degradation curves and the dashed lines are the first derivative of the degradation curves.

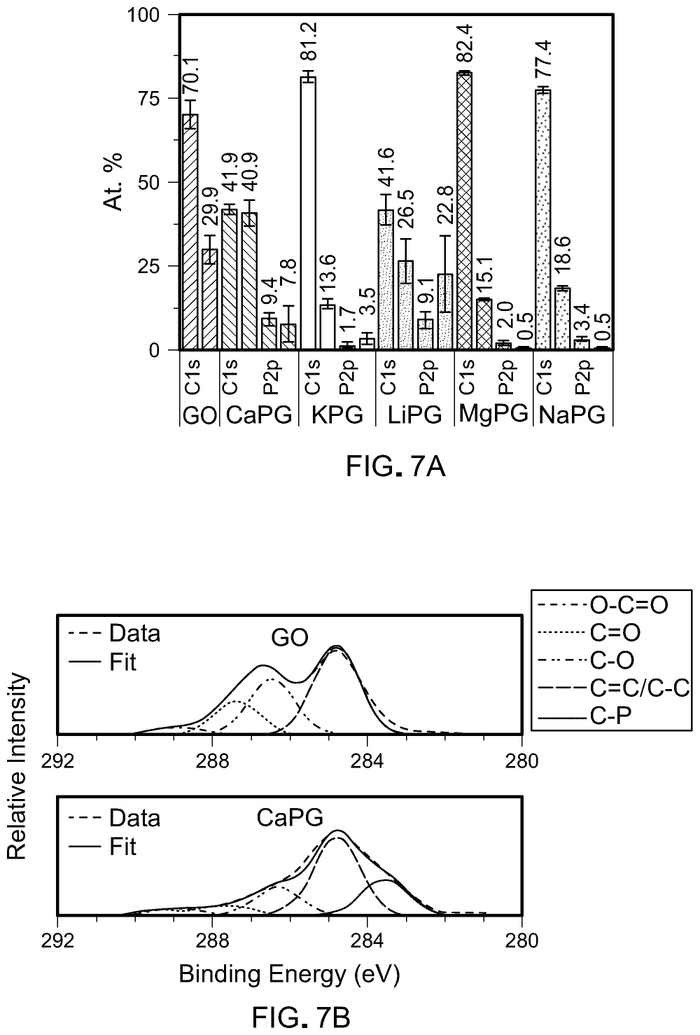

XPS was performed to quantify the atomic composition of X-PG material with various counter ions (Ca²⁺, K⁺, Li⁺, Mg²⁺, or Na⁺). Referring to FIG. 7A, analysis of the XPS spectra of the phosphate modified GO materials showed that both phosphate and the counter ion were effectively incorporated onto the GO scaffold. Furthermore, referring to FIG. 7B, the emergence of a new peak at 283.5 eV corresponds to P—C bonds, thus confirming the covalent tethering of phosphates to graphene oxide. These XPS results demonstrate that cationic inducerons can be successfully incorporated into graphene oxide via phosphate modification to impart bioactivity to these materials.

Based on the synthetic mechanism for phosphate modification of graphene oxide, the maximum counter ion-to-phosphorus (X:P) ratio is two. The phosphate modified graphene oxide materials characterized in these XPS experiments have an X:P ratio less than two. Without being bound by theory, it is believed that this lower X:P ratio may be due to ion exchange with water or incomplete removal of ethyl substituents from triethyl phosphite as it is incorporated into the phosphate backbone.

The kinetics of the modified Arbuzov reaction for PG synthesis were also studied. X-PG synthesis was conducted on a 500 mg graphene oxide scale using the procedure described in Example 1. Approximately 20 mL intrasample aliquots were collected via a syringe as the reactions progressed at 160° C. The reactions were heated from room temperature to 160° C. over a period of 60 min, and time points were collected for each material (0−, 0+, 1, 2, 4, 8, 12, 16, 24, 36, 48, 60, and 72 h, where 0-h was at room temperature before heating and 0+h was as soon as the reaction reached 160° C.). Time points were washed with tetrahydrofuran and centrifuged at 3,600×g for 5 minutes. Supernatants were discarded and pellets were re-dispersed in fresh solvent for additional wash steps. Pellets were washed four additional times with tetrahydrofuran and dried under vacuum until dry.

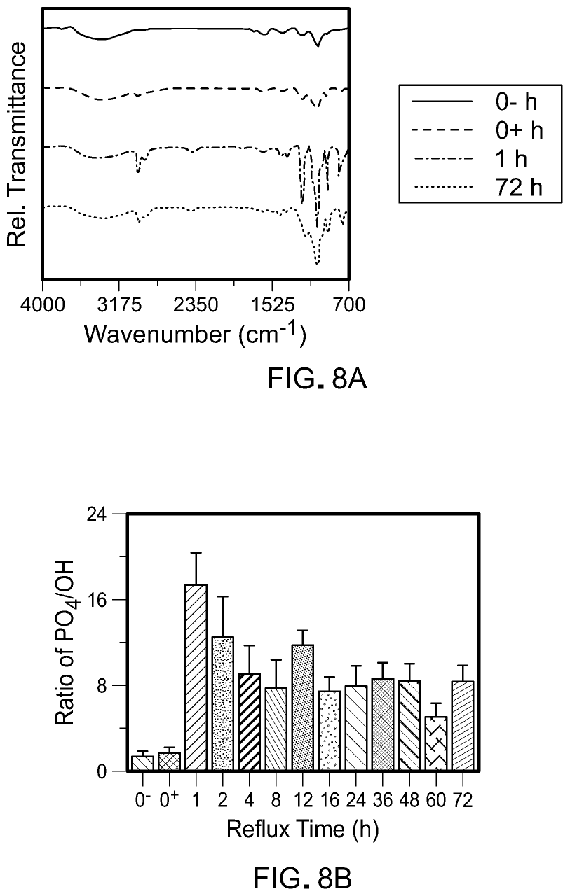

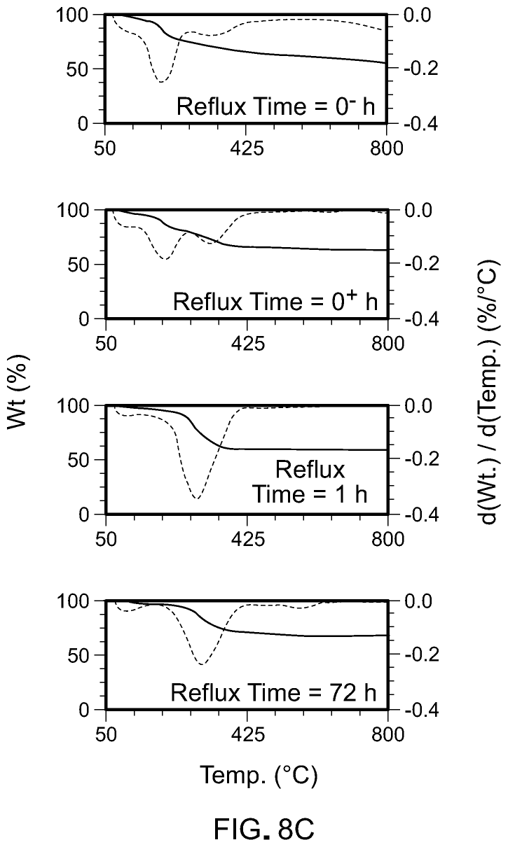

FIGS. 8A-8C show FTIR and TGA for CaPG as the modified Arbuzov reaction progressed. FIG. 8A shows FTIR spectra for CaPG as a function of reaction progression. FIG. 8B shows the ratio of phosphate-to-hydroxyl stretches from the maximum peak intensity obtained from FTIR for CaPG as a function of reaction time. After 1 hour at reflux at 160° C., FTIR showed the emergence of phosphate stretches. Qualitative evaluation of normalized FTIR spectra for all CaPG time points tested also showed that the intensity of the phosphate-to-hydroxyl stretches reached a maximum after 1 hour at reflux and tapered as the reaction progressed. FTIR of PG materials with other cations also showed phosphate functionalization occurring within the first few hours of the reaction. FIG. 8C shows TGA for selected time points for CaPG. TGA agreed with the qualitative FTIR analysis, suggesting phosphate functionalization primarily occurred early during the reaction.

### Example 3—Preparation and Chemical Characterization of Solid X-PG Constructs

X-PG powders were subjected to material processing techniques to generate solid X-PG constructs, referred to in the context of these examples as X-PG pellets. X-PG powders were dried for 24 h under high vacuum prior to material processing. A custom stainless steel mold, with an inner diameter of 3.75 mm, was heated to 200° C. in a Fischer Isotemp vacuum oven. After heating, the mold was removed from the oven and approximately 20-25 mg PG powder was immediately added. The powder was pressed for 1 min on a Columbian D63 ½ bench vise and then removed from the mold as a pellet. Pellets were then heat treated at 200° C. for 20 min. Graphene oxide constructs were not subjected to heat treatment since heat treatment can destroy the structural integrity of the pellets. The pellets had an average diameter of 3.75 mm and an average thickness of between 1 and 2 mm.

FTIR and TGA confirmed that the processing did not degrade the covalent phosphate functionalization of the graphene oxide. Specifically, FTIR confirmed that pellet formation had minimal effect on surface functionalization of X-PG materials. TGA of pellet cross sections showed that functionalization on the interior of the pellets was also minimally affected by the processing.

Raman spectroscopy was performed to investigate the internal structure of the pellets. Raman spectroscopy clearly identified the D, D′, and D+D′ bands that arise from a highly functionalized graphenic backbone, and the intensities of these “disorder” modes normalized to the G-band that originates from the sp² hybridized backbone were not substantially altered via processing into pellets or upon exposure of pellets to water. The G-band peak location of the pellets relative to that of graphite was found to shift slightly (−2 meV) for CaPG and MgPG upon pressing into pellets which may suggest that the graphenic backbone is in a different mechanical environment, although the shift is only ˜0.1·kBT. For most powdered materials, the G′ mode was accurately fitted by two Lorentzian functions in a form that indicated an ordered bulk graphenic material. However, LiPG had a more prominent (G′)₂ peak compared to graphite and other X-PG materials, indicating a change in the electronic and/or phonon structure of its graphenic backbone that may be related to functionalization and/or exfoliation. The intensity of the (G′)₂ peak relative to the (G′)₁ peak generally increased upon hot pressing of powders into pellets and upon water exposure; however, the broadness of the peaks resulted in uncertainties in the fits that are too large to enable an accurate determination of state based on the G′ mode.

Analysis of X-ray powder diffraction (XRD) spectra confirmed that the phosphate functionalization remained intact through pellet processing, but revealed differences in interplanar spacing for the different counter ions. The X-PG pellets possessed a broad peak from 20-30 20 (0.44-0.30 nm); however, LiPG had other peaks at shorter 20 (1.35, 1.17 and 0.85 nm), suggesting bulky functionalization. After exposure to water, the XRD spectra changed for the pellets, with the trend toward larger spacing for all PG materials other than LiPG for which the shorter 20 peaks were absent. Without being bound by theory, it is believed that upon exposure to water, water molecules may intercalate the graphenic layers, increasing spacing, and for LiPG, the material becomes more dispersed, in agreement with the macroscale observation that the LiPG pellets are not water stable (discussed below in Example 5).

### Example 4—Mechanical Properties of Solid X-PG Constructs

Mechanical properties of X-PG pellets fabricated as described in Example 4, including density, porosity, hardness, and compressive and shear moduli, were characterized. Generally, these properties were observed to be comparable to those of hard tissue, such as trabecular bone. Furthermore, the compressive mechanical properties of X-PG pellets did not display strain-rate dependence, indicating that these materials used as bone scaffolds can withstand a variety of loads without comprising mechanical integrity.

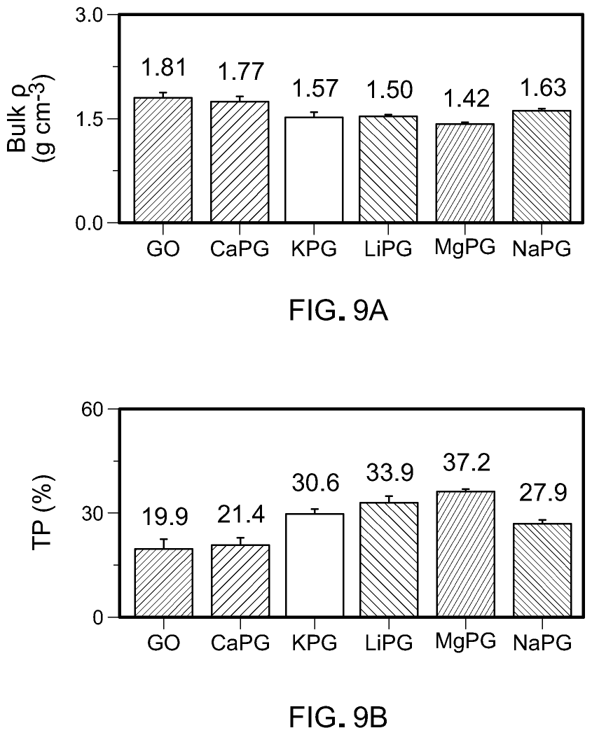

Bulk density of graphene oxide and X-PG pellets was obtained using the mass and cylindrical dimensions of the constructs. Total porosity was calculated using the theoretical density (2.26 g/cm³) of graphite. Referring to FIGS. 9A and 9B, as compared to solid constructs of graphene oxide, X-PG pellets had a lower bulk density (FIG. 9A) and a higher total porosity (FIG. 9B). Without being bound by theory, it is believed that the lower density and higher porosity of X-PG pellets may be due to phosphate functionalization, which increases the interlayer distance between sheets of graphene oxide. The hardness of X-PG pellets, as measured by Vickers Microhardness (MHV) testing, was found to be generally independent of the identity of the counter ion functionalization.

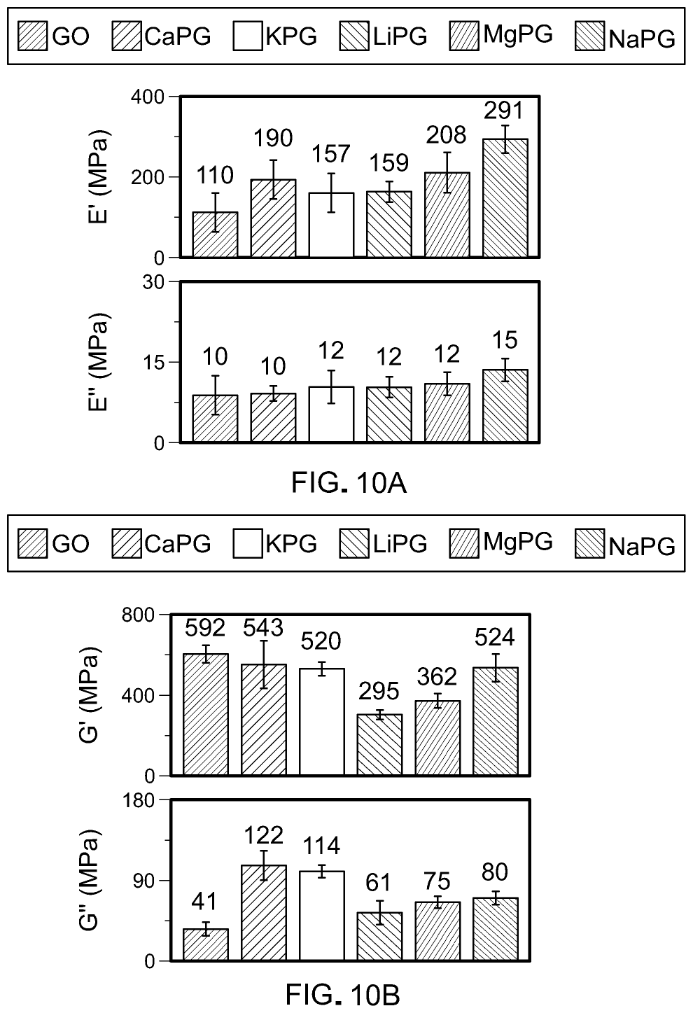

The compressive and shear mechanical properties of X-PG pellets were evaluated using dynamic mechanical analysis (DMA). FIG. 10A shows storage moduli (E′) and loss moduli (E″) for graphene oxide and X-PG pellets with various counter ion identities as measured by DMA. The storage moduli E′ of the X-PG pellets was on the order of that of hard tissue. FIG. 10B shows shear storage moduli (G′) and shear loss moduli (G″) for X-PG pellets with various counter ion identities as measured by viscoelastic torsional shear testing.

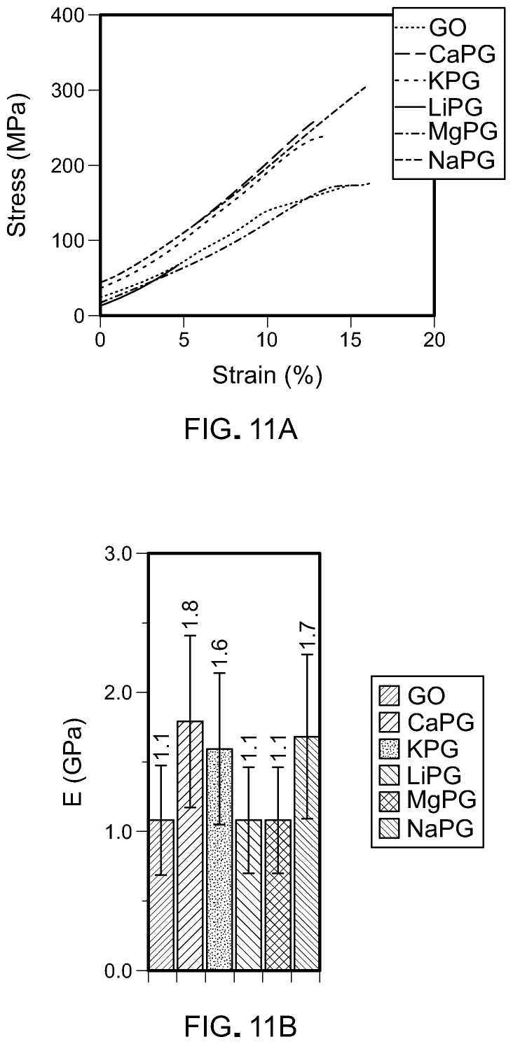

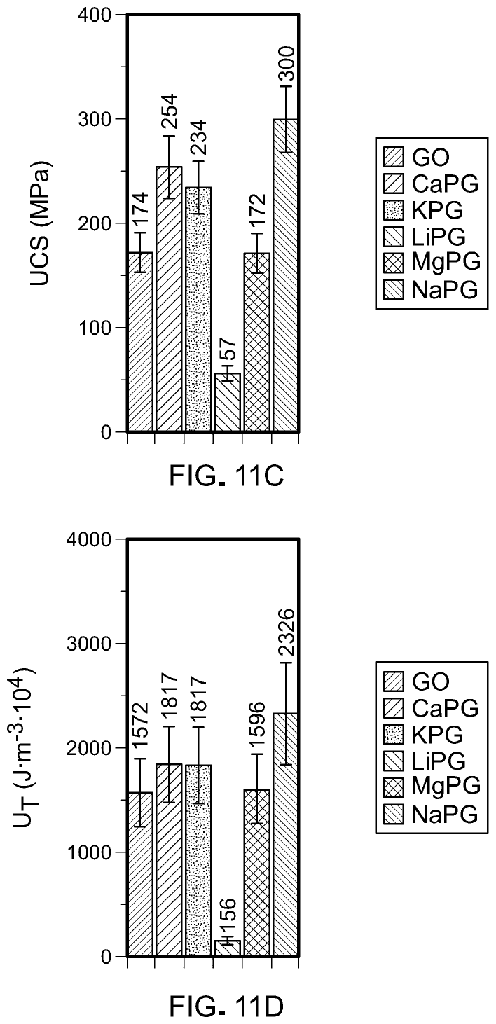

Referring to FIGS. 11A-11D, compressive universal testing of X-PG pellets was performed with a load cell of 50 kN at strain rates of 0.001, 0.01, and 0.1 s⁻¹ until construct failure. FIG. 11A shows X-PG stress-strain curves measured at strain rate of 0.1 s⁻¹. Analysis of the X-PG stress strain curves demonstrated that the constructs were tough (FIG. 11D) with high ultimate compressive strengths (FIG. 11C) and Young's moduli E (FIG. 11B). Universal testing demonstrated that the compressive mechanical properties of X-PG pellets did not have a significant dependence on strain rate.

### Example 5—Water Stability of Solid PG Constructs

X-PG pellets were exposed to aqueous conditions and their storage modulus (E′) and loss modulus (E″) values were evaluated as a function of time to characterize the ex vivo water stability of the constructs.

X-PG pellets were submerged in 1 mL of 1× phosphate buffered saline (PBS) equilibrated to 37° C. in 48 well cell culture plates. Samples were stored at 37° C. in a MyTemp Mini Incubator (Benchmark Scientific) incubator for the duration of the experiment. DMA was performed using a sand blasted 8 mm geometry. Zero time point DMA measurements were measured immediately after the constructs were submerged in PBS. Subsequent time point DMA measurements were taken and liquid volume was replenished with DI water as needed.

Hydrated X-PG pellets at the zero time point had an E′ an order of magnitude lower than dry pellets. Over a period of 28 days, minimal changes in E′ values were observed. Specifically, the compressive modulus of CaPG changed by 40±18%; the compressive modulus of KPG changed by 44±11%; the compressive modulus of MgPG: changed by 59±10%; the compressive modulus of NaPG changed by 52±18%; and the compressive modulus of LiPG changed by 100%. A similar trend was observed in E″ values. No data was collected for graphene oxide because the graphene oxide pellet did not survive beyond day 1.

At the conclusion of the ex vivo stability test, intact X-PG pellets were frozen at −80° C., lyophilized until dry, and then subjected to DMA to characterize the mechanical integrity of the hydrated constructs. Compared to pellets that had not been hydrated, the DMA compressive moduli and torsional shear moduli of the hydrated pellets decreased by an order of magnitude. There were also significant decreases in the bulk density and increases in the total porosity for pellets of CaPG and LiPG; however, there were no changes for KPG, MgPG, and NaPG pellets. Changes for LiPG pellets were likely a result of a loss of mechanical integrity that was observed after the ex vivo experiment.

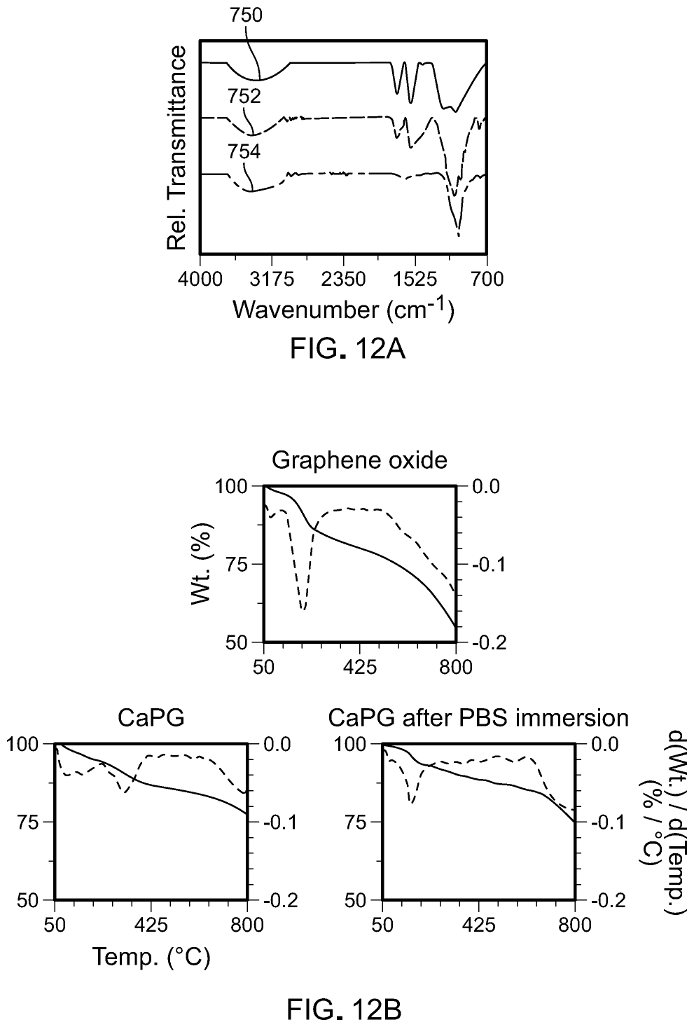

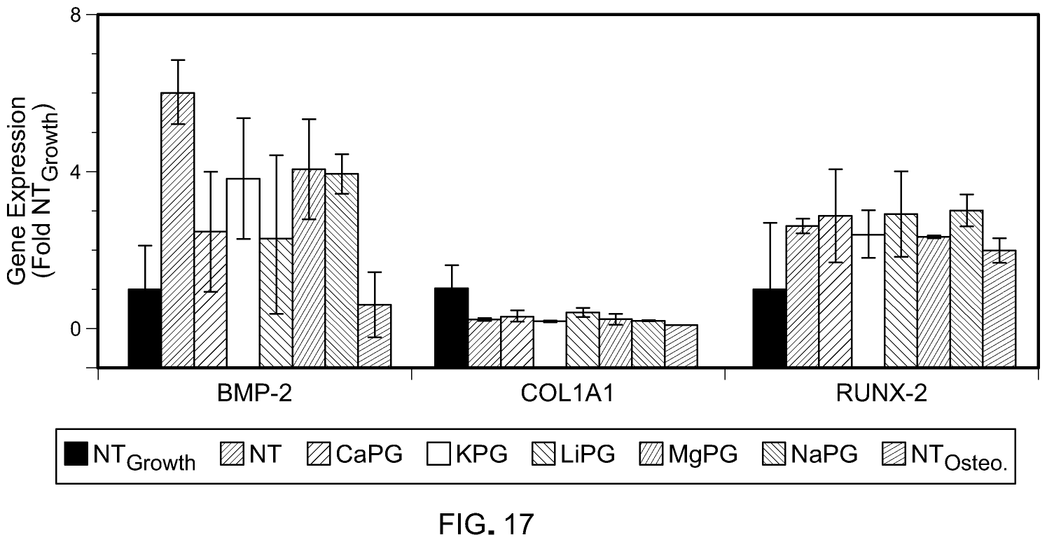

After 28 days of hydration, the chemical composition of the X-PG pellets was evaluated with FTIR and TGA. Referring to FIGS. 12A and 12B, chemical changes were observed in CaPG pellets. FIG. 17A shows FTIR spectra for dry graphene oxide 750, a dry CaPG pellet 752, and a CaPG pellet after 28 days of PBS immersion. After hydration, the FTIR spectrum (FIG. 12A) and TGA thermogram (FIG. 12B) of CaPG pellets were similar to those of graphene oxide. No significant changes were observed in X-PG pellets with other counter ions.

To investigate the chemical changes in CaPG, a calcium elution study was performed to quantitatively measure calcium elution from CaPG pellets into PBS as a function of time. Graphene oxide pellets and CaPG pellets were submerged in 1 mL of 1× PBS in 15 mL centrifuge tubes at 37° C. Time points were obtained by aliquoting 20 μL of sample. Calcium quantification was determined using a colorimetric assay with ocresolphthalein complexone chelator. Reagent 1 contained 0.3 mol/L of 2-amino-2-methyl-1-propanol (Alfa Aesar) and adjusted to pH 10.5. Reagent 2 contained 0.16 mmol/L of o-cresolphthalein complexone (Alfa Aesar) and 7.0 mmol/L of 8-hydroxyquinoline (Alfa Aesar). Reagent 1 (145 μL), Reagent 2 (145 μL), and sample of interest (2.9 μL) were added to 96 well cell culture plates and incubated at room temperature for 10 min. Absorbance was measured at 578 nm on a microplate reader.

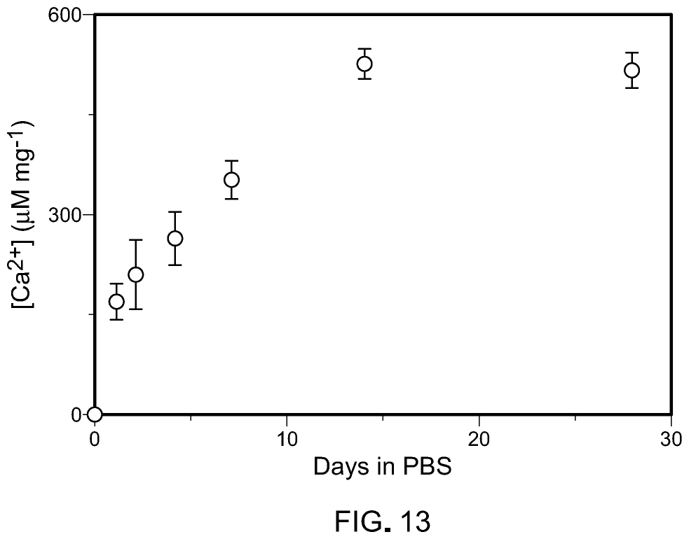

The calcium release profile as a function of time is shown in FIG. 13, with calcium concentration values normalized to pellet mass. The CaPG pellets remained intact throughout the experiment, with no observable defects or swelling after the conclusion. By fitting the calcium ion elution measurements to a mathematical model, it was concluded that the elution of calcium from the CaPG pellets was diffusion controlled.

Similar analysis was also conducted on LiPG and MgPG pellets. Unlike CaPG constructs, LiPG and MgPG pellets displayed no measurable cation elution from the pellets over a period of 28 days, which agrees with the chemical analysis of pellets (FTIR and TGA). Without being bound by theory, it is believed that since cation elution is controlled by a diffusion mechanism, the monovalency of lithium may not have established a sufficient electrochemical gradient in PBS to facilitate lithium diffusion in quantifiable concentrations. In the case of MgPG pellets, lack of magnesium elution may have been due to several factors, such as the low electropositivity and diffusivity of magnesium that could prevent measurable magnesium diffusion.

### Example 6—Cytocompatiblity and Cell Growth on X-PG Materials

The particle size of a biomaterial can sometimes be correlated with the cytocompatibility of the biomaterial, where larger particle sizes are generally more cytocompatible than smaller particles. The size distribution of graphene oxide and X-PG particles was evaluated by drop casting dispersions of the materials in DI water at a concentration of 50 μg/mL onto glass microscope slides and measuring using dynamic light scattering (DLS) and direct optical imaging. Graphene oxide particles and X-PG particles were observed to be generally similarly sized, on the order of several microns in diameter.

The Zeta potential of dispersions of graphene oxide and X-PG particles was also measured and demonstrated that the materials good stability in water and that flocculation had minimal effect on the particle size analysis.

The cytocompatibility of X-PG materials was studied to investigate the potential for the use of X-PG materials in biomedical applications. NIH-3T3 fibroblasts and RAW 264.7 macrophages were used, because fibroblasts are an important cell type in wound healing, macrophages are an important cell type of the immune system, and both cells lines are widely investigated, allowing for direct comparisons to other studies. Cells were exposed to dispersions of X-PG materials diluted in their cell culture media and their vitality was assessed after 2 days. Specifically, powdered X-PG materials were suspended in sterile DI water at concentrations of at least 1 mg mL⁻¹ and sterilized via exposure to 254 nm ultraviolet light for 10 min. For the counter ion cytocompatibility experiment, the anion associated with each cation was chloride, and the cellular exposure concentrations were based on the mass concentration of the cation. For the PG materials, the cellular exposure concentration was based on the total mass of the PG material. These dispersions were diluted to the final, indicated concentration in complete cell culture media.

NIH-3T3 fibroblasts and RAW 264.7 macrophages were seeded in the interior wells of 96-well plates at a density of at 3×10⁴ and 2×10⁴ cells cm⁻². After 8 h, the cells were well adhered, and the media was exchanged for media containing the experimental samples. Since different exposure concentrations involved different volumes of the stock suspensions of PG materials, DI water was added as appropriate to ensure that all wells were diluted by the same volume. Control cells were exposed to DI water at the same volume. The final dilution of cell culture media was <2% v/v. Cells were allowed to grow for 48 h, and then they were subjected to the vitality assays.

Vitality assays included assessments of cellular proliferation, metabolism, and death using fluorescent reporters. To do so, the cell culture media that contained the experimental samples was aspirated and the cells were washed with PBS (#10010049, ThermoFischer Scientific). The washed cells were exposed to 20 μM of Hoechst 33342 (#62249, ThermoFischer Scientific), 5 μM of Calcein AM (#PK-CA707-80011-2, PromoKine), and 2.5 μM of ethidium homodimer-1 (#L3223, ThermoFischer Scientific) for 15 minutes. Hoechst 3342 labels the DNA of cell nuclei and then becomes brightly fluorescent, reporting proliferation. Upon cellular internalization of Calcein AM, it is converted to a fluorescent form by esterases, reporting metabolism. Ethidium homodimer-1 becomes brightly fluorescent upon binding DNA but is excluded from the nuclei of live cells, thus reporting dead cells. To quantify fluorescence of these molecules, a fluorescence microplate reader was used with excitations of 350/20 nm, 483/20 nm, and 525/20 nm and emissions of 461/20 nm, 525/20 nm, and 617/20 nm for Hoechst 33342, Calcein AM, and ethidium homodimer-1, respectively. Since graphenic materials may alter fluorescence assays, direct fluorescence imaging was also performed.

Vitality analyses revealed that X-PG materials were cytocompatible, with cellular exposure up to 100 μg mL⁻¹ having no significant effect on proliferation or metabolism, although there were some small but significant increases in the percent dead macrophages for LiPG and MgPG. The maximum concentration of X-PG materials was limited to 100 μg mL⁻¹ since beyond that concentration the graphenic materials begin to substantially cover the cells, artificially reducing vitality.

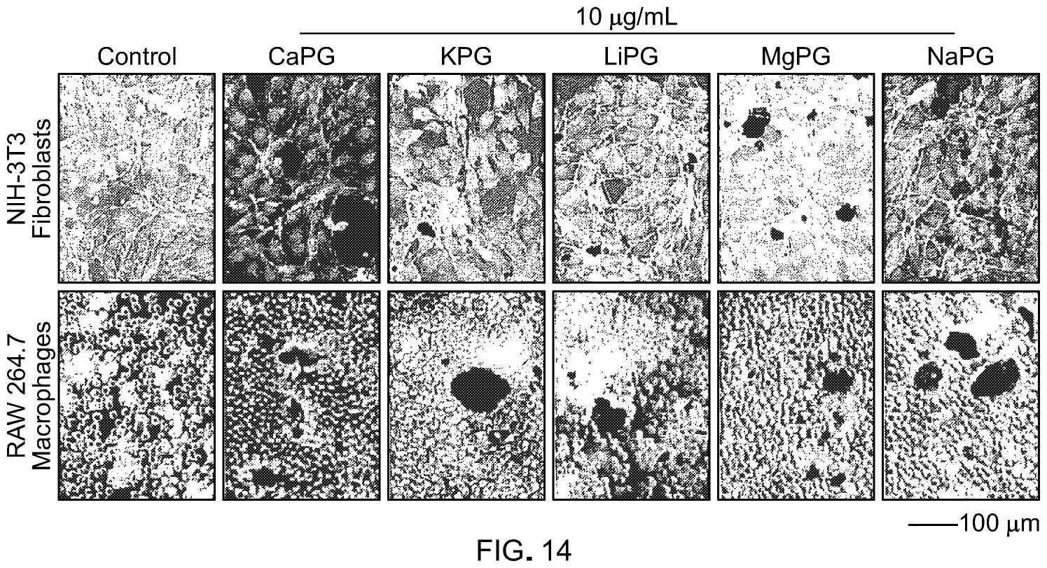

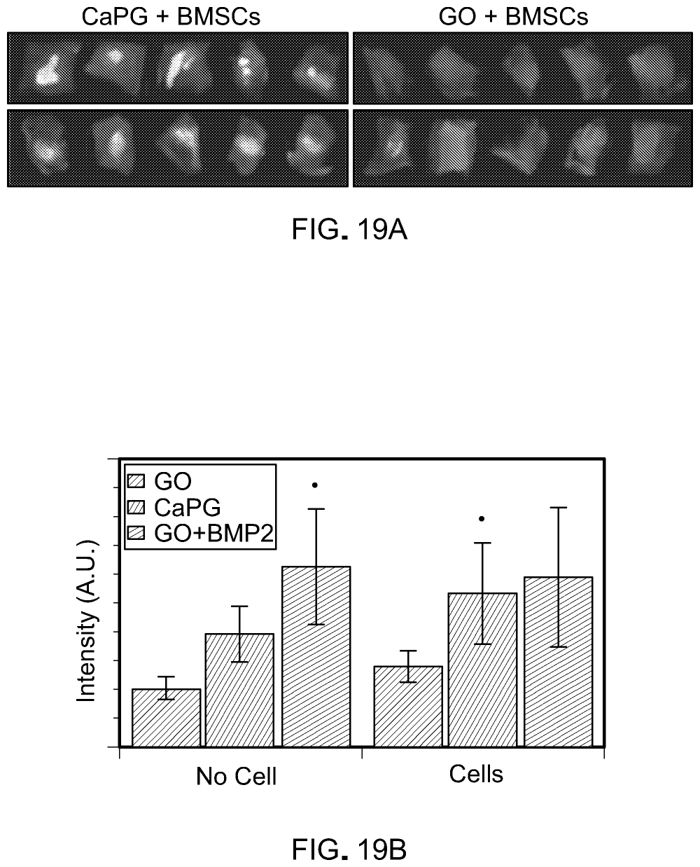

Referring to FIG. 14, to further confirm cytocompatibility, selected sub-cellular compartments were labeled and imaged, including nuclei (the genomic center of the cell), filamentous actin (a major component of the cytoskeleton that is involved in numerous critical cellular processes), and mitochondria (the metabolic center of the cell). Imaging confirmed that the PG materials did not alter these sub-cellular compartments, and cells were able to adhere to and interact with flocculants without deleterious effects. In FIG. 19, blue regions are Hoechst 3342 labeling nuclei, green is Acti-stain™ 488 phalloidin that labels filamentous actin, and red is MitoTracker™ that labels mitochondria. The dark regions are X-PG materials.

Since the counter ions are bioinstructive, the cytocompatibility of high concentrations of the cations was also tested. The cations were cytocompatible up to a cation concentration of 125 μg mL⁻¹, at which point lithium significantly reduced cellular vitality. Since XPS demonstrated that the counter ions were less than 10 wt. % of the X-PG materials, a cation concentration of 125 μg mL⁻¹ would correspond to a total X-PG concentration of ˜1250 μg mL⁻¹ based on wt. %, but since the cations are associated with the polyphosphate and are controllably released over time, a larger X-PG concentration would be needed for a free cation concentration of 125 μg mL⁻¹.

To assess cell growth on X-PG materials, NIH-3T3 fibroblasts were seeded on substrates of X-PG materials prepared by drop casting concentrated dispersions of X-PG material onto microscopy coverslips. The water was allowed to evaporate, creating a layer of X-PG material on the coverslips. The substrates were sterilized by immersion in 70% ethanol for 10 min, followed by aspiration and washing three times with PBS. During these steps, loose material was dislodged. Coverslips containing regions of X-PG substrates were placed into cell culture dishes, and NIH-3T3 fibroblasts were added to the entire dish and cultured for 24 h. After 24 h, the cells were exposed to a labeling solution including Hoechst 33342 and Calcein AM to enable assessment of cellular proliferation, metabolism, and death using fluorescent reporters. The labeling solution was aspirated, the cells washed with PBS, and fixed with 3.7% formaldehyde for 10 min. After fixation, the cells were washed and the coverslips mounted onto microscopy slides for confocal imaging. Confocal imaging also demonstrated that cells adhered to and grew on top of regions of X-PG materials, suggesting X-PG materials have potential for in vivo tissue engineering applications.

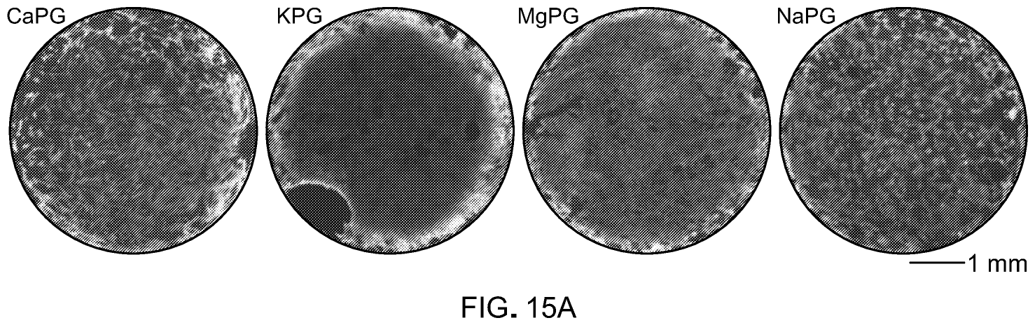

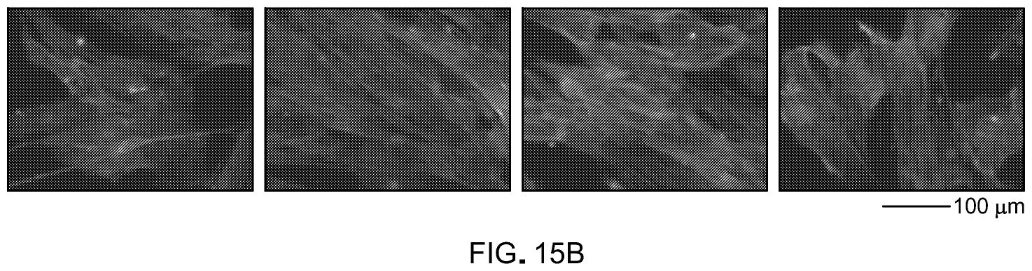

To evaluate the potential of solid X-PG constructs as tissue engineering scaffolds, a highly potent cell type, hMSCs, was cultured directly on X-PG pellets. After 7 days, the proliferation, morphology, and important sub-cellular compartments of the cultured cells were evaluated. FIGS. 15A and 15B show whole-construct images and higher-magnification images of hMSCs on X-PG pellets after the 7 day culture. The higher magnification images of FIG. 15B shows normal, well-defined structures of DNA of the nucleus (blue), F-actin (green), and mitochondria (red).

As can be seen from the varying density of hMSCs in FIG. 15A, hMSCs proliferated at different rates and had different morphologies based on the X-PG counter ion. Without being bound by theory, it is believed that this may be due to release of inducerons and to the interfacial topology of the construct. Evaluation of sub-cellular compartments revealed the presence of well-defined F-actin structures, organized mitochondrial tracks, and prominent nuclei with relatively homogeneous distribution of DNA within the nucleus. Since graphene oxide and LiPG pellets are not water stable, hMSCs were not evaluated for these materials. Overall, X-PG pellets facilitated the growth of highly potent cells, and the pellets with different counter ions have potential to direct stem cells.

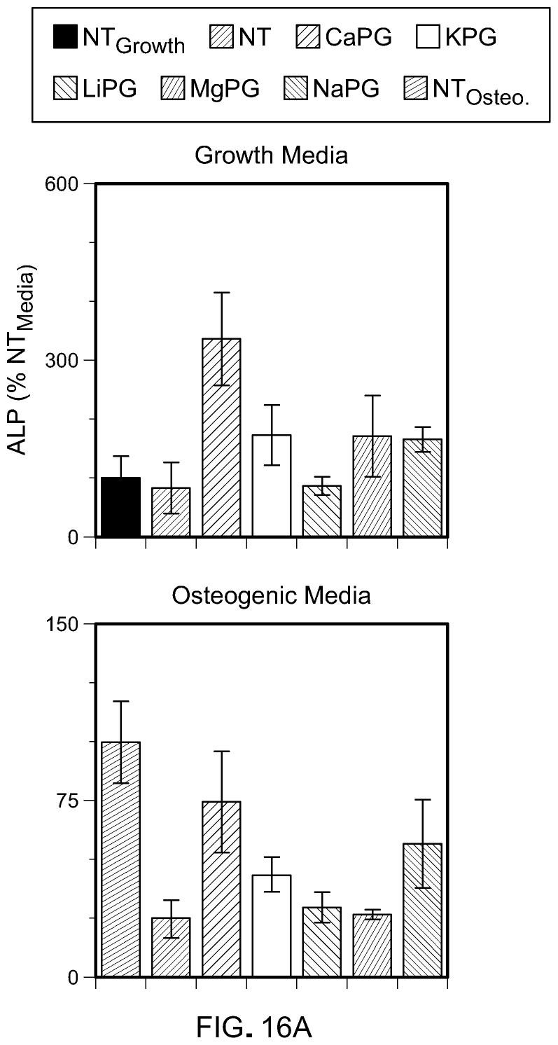

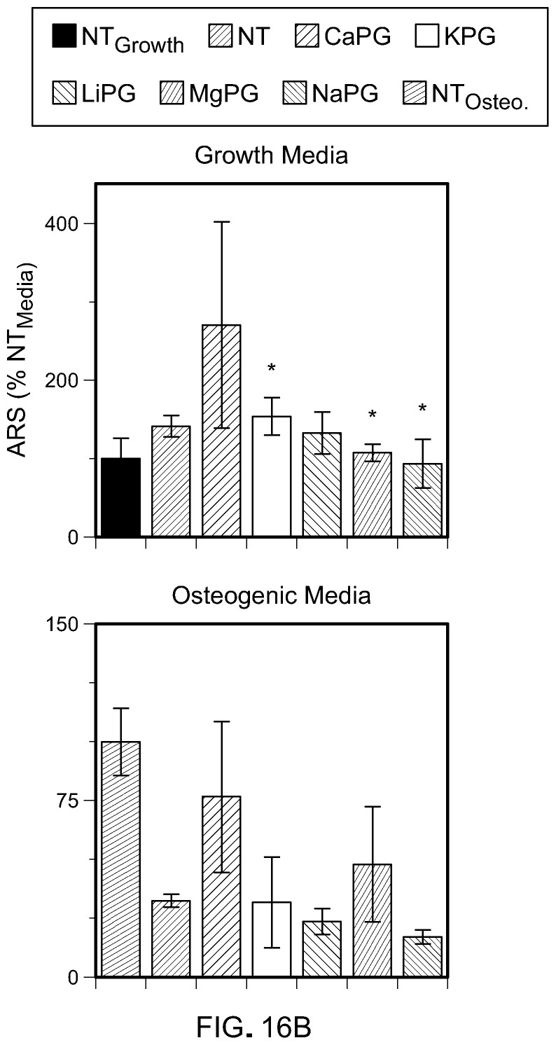

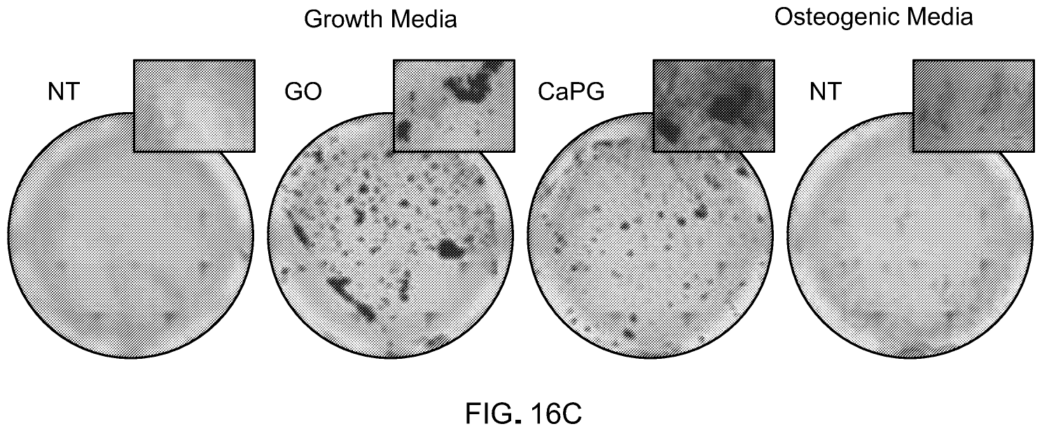

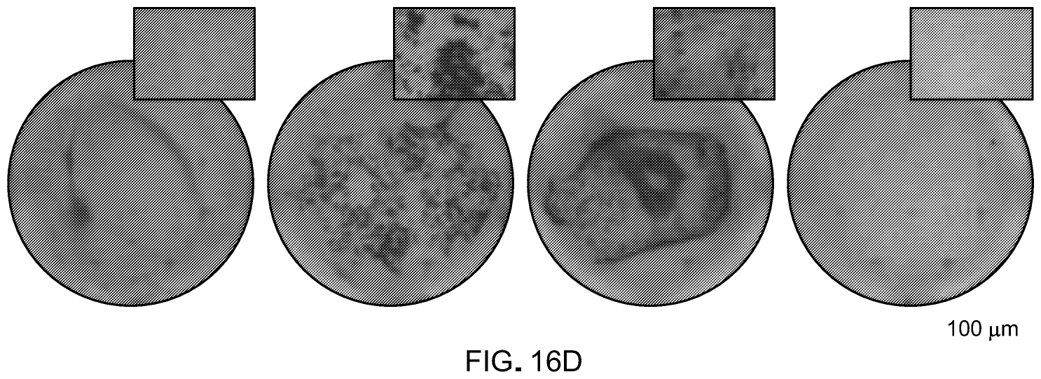

Cellular exposure to CaPG resulted in hMSCs differentiating towards an osteoblastic phenotype. Referring to FIGS. 16A and 16C, alkaline phosphatase (ALP) is highly expressed in osteoblasts, and when hMSCs that were cultured in growth media designed to maintain multipotency were exposed to CaPG, there was a 240% increase in ALP expression. FIG. 16A shows ALP expression of hMSCs exposed to X-PG materials for 10 days for growth media and 21 days for osteogenic media. FIG. 16C shows whole-well and higher-magnification images of ALP expression, shown in red. Referring to FIGS. 16B and 16D, a similar result was obtained when assaying for Alizarin Red S (ARS) that labels calcium deposits that are indicative of mineralization from cells displaying an osteogenic phenotype: hMSCs exposed to CaPG had a 170% increase in the intensity of the ARS labeling. FIG. 16B shows ARS expression of hMSCs exposed to X-PG materials for 28 days, quantified from ARS absorbance. FIG. 16D shows whole-well and higher-magnification images of ARS expression, shown in red. When hMSCs were cultured in commercially available media that is designed to inspire osteogenic differentiation, cells exposed to X-PG materials other than CaPG had similar expression levels to the osteogenic media control while the levels were reduced for the other X-PG materials.

Reverse transcription quantitative polymerase chain reaction (RT-qPCR) was used to quantify the expression of important osteogenic genes of hMSCs exposed to X-PG materials: collagen type I alpha 1 (COL1A1), bone morphogenetic protein 2 (BMP-2), and runt-related transcription factor 2 (RUNX-2). Small nuclear ribonucleoprotein D3 (SNRPD3) was used as the reference gene due to its constant level of expression. FIG. 17 shows gene expression after 14 days of exposure to X-PG materials, as quantified from RT-qPCR. Cells exposed to X-PG materials had levels of gene expression similar to the osteogenic media positive control and significantly different than the growth media negative control for COL1A1. While there were increases in the expression of BMP-2 and RUNX-2 for hMSCs exposed to X-PG materials, there was variability in the measurements that may be due to the presence of PCR inhibitors.

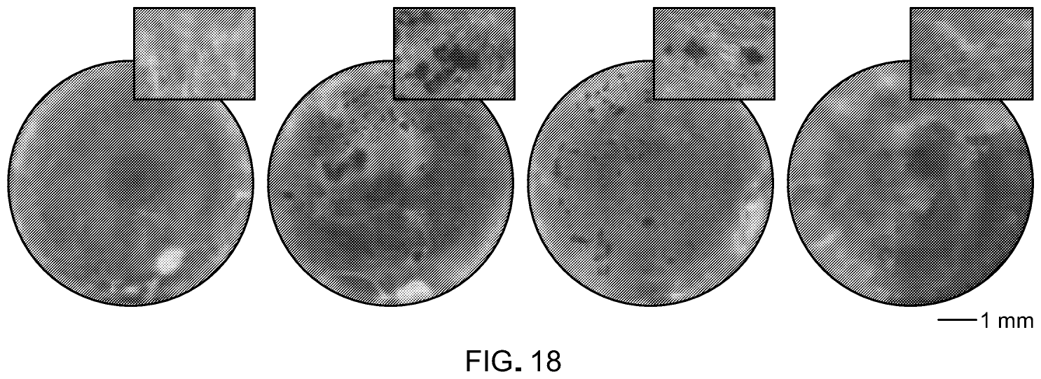

Association of the X-PG materials with the hMSCs was substantial. After 3.5 days of exposure to an initial concentration of 100 μg mL⁻¹, the average concentrations of PG materials remaining in growth and osteogenic media were 4.5±2.2 μg mL⁻¹ and 14.4±3.6 μg mL⁻¹, respectively, corresponding to a cellular association of 3.2±0.5 ng cell⁻¹ and 11.4±7.1 ng cell⁻¹, which is significantly higher than the reported uptake of single wall carbon nanotubes in hMSCs. Even 21 days after an initial exposure to 100 μg mL⁻¹ followed by fresh media changes every 3.5 days, a substantial amount of X-PG materials remained incorporated into the cellular environment without any observed negative cellular effects. Referring to FIG. 18, representative whole-well and higher magnification images of nuclei (blue), F-actin (green), and phase contrast (gray) show the incorporation of X-PG materials in the cellular environment.

Overall, of the X-PG materials studied, CaPG was best able to induce hMSCs to differentiate into an osteoblastic phenotype. Without being bound by theory, it is believed that CaPG mimics natural bony apatite and CaPG pellets controllably release calcium ion inducerons over time that can stimulate differentiation, thus inspiring differentiation. Even for hMSCs maintained in growth media designed to preserve multipotency, CaPG resulted in significant osteogenic differentiation.

### Example 7—CaPG Inducement of Osteogenesis in Mice

Animal studies were performed to investigate the osteogenic properties of X-PG in mice. Col3.6 fluorescent protein reporter mice expressing two distinct fluorescent proteins (topaz and cyan) were used to understand how the presence of calcium and phosphate ions can contribute to new bone formation. Col3.6 mice contain a 3.6-kilobase DNA fragment derived from the rat type I collagen (Col1α1) promoter that drives strong expression of fluorescent proteins in pre-osteoblasts and osteoblasts hence identifying bone tissues and allowing for an in-depth characterization of bone formation at the cellular level. By using Col3.6Topaz mice as host and bone marrow stromal cells (BMSCs) from Col3.6Cyan mice as donor cells, the contributions of each cell during bone formation can be distinguished based on their distinct fluorescent proteins.

A dose of 0.54 mg of either graphene oxide or CaPG material (approximately 20 mg/kg per mouse) in 50 μl of PBS was injected subcutaneously (two injections per mouse) into 11-week-old Col3.6Topaz and NSG/Col3.6Topaz mice. The Col3.6Topaz mice received injections of material alone. The NSG/Col3.6Topaz immunodeficient mice received injections of graphene oxide or CaPG material mixed with 1×10⁶ bone marrow stromal cells that were isolated from Col3.6Cyan mice a week prior and cultured in vitro. Control groups received 2.5 μg of rhBMP-2, known to be a strong inducer of bone growth, mixed with graphene oxide prior to injections. The injected material formed a coalesced mass of particles resembling a macroscopic implant. One day prior to sacrifice, alizarin complexone at a dose of 30 mg/kg was injected intraperitoneally to mark areas of active mineralization.

At 8 weeks, the mice were euthanized and the subcutaneous tissue in and around the implants were dissected and fixed in 10% formalin. Radiographs of explanted tissues were acquired using a digital X-ray system (Faxitron LX-60) at 1× magnification. The tissues were then cryosectioned and transferred to glass slides using tape transfer process. The sections were initially imaged for DIC, fluorescent reporters and AC, and then sequentially stained and imaged for TRAP, ALP and DAPI, and toluidine blue.

Referring to FIG. 19A, radiographs of the implant and surrounding tissue revealed mineralized tissue formation for mice that received CaPG with bone marrow stromal cells (referred to as CaPG+BMSC), but not for mice that received graphene oxide with bone marrow stromal cells (GO+BMSC). Referring to FIG. 19B, quantification of X-ray image intensity, which corresponds to quantification of the mineralized tissue, revealed that the level of bone formation for mice with CaPG+BMSC was significantly increased compared to that for mice with GO+BMSC, and was statistically similar to the BMP2 positive control that is known to strongly induce bone formation.

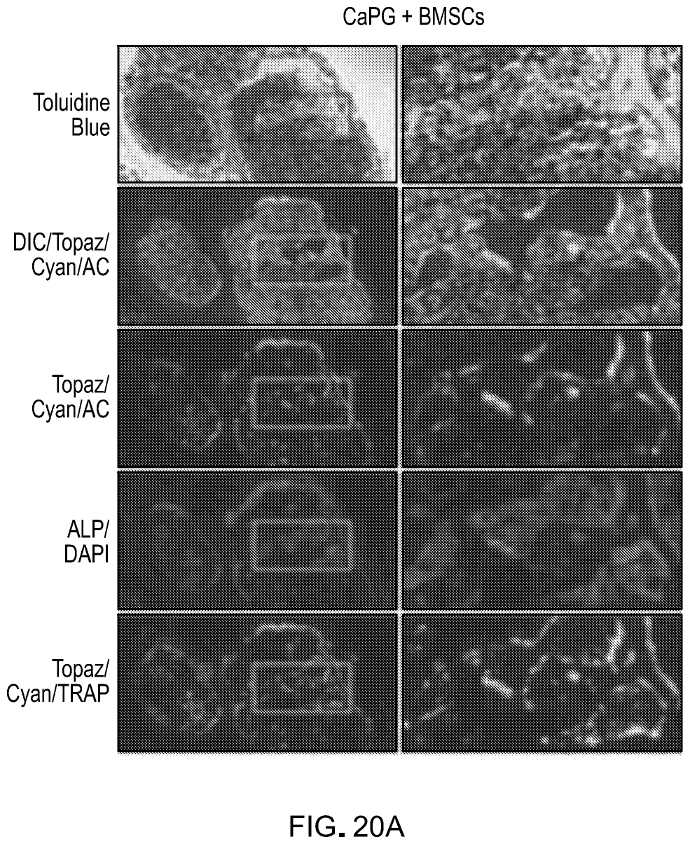

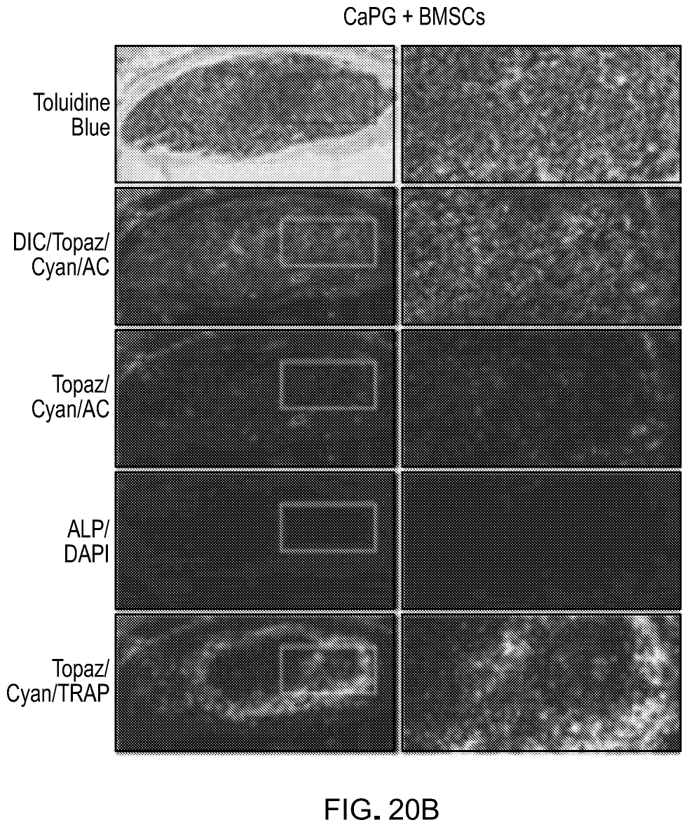

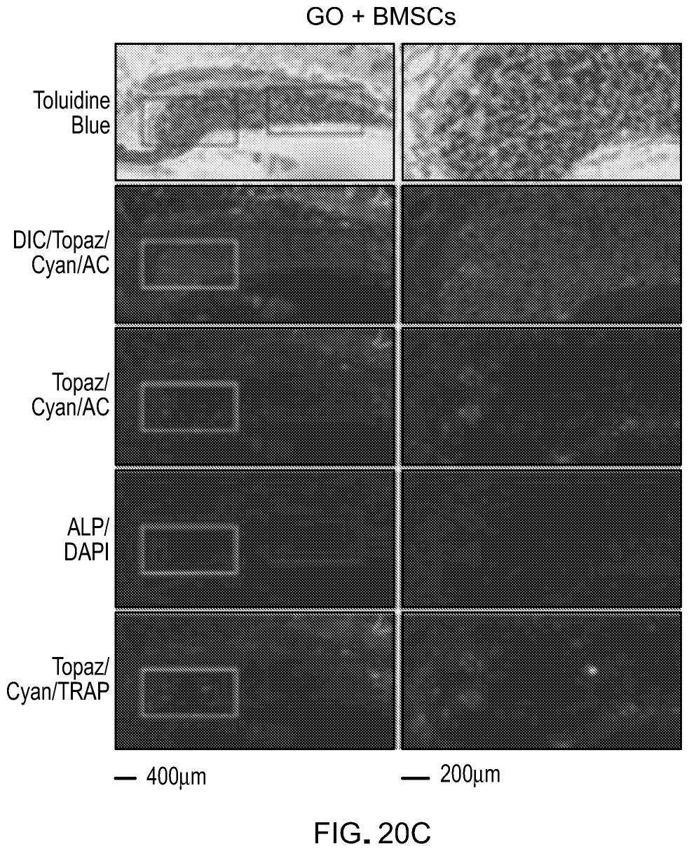

Referring to FIGS. 20A-20C, histological evaluation was performed on toluidine blue and overlay images for various signals for tissue exposed to CaPG+BMSC (FIG. 20A), CaPG+BMSC (FIG. 20B), and GO+BMSC (FIG. 20C). Overlay images for darkfield (DIC), donor cells (cyan), host cells (topaz), alizarin complexone (AC, red), alkaline phosphatase (ALP, red), DAPI (blue), and TRAP (yellow) are shown. Mineral accumulation is shown in white in DIC images. Host-derived and donor-derived osteoblasts are expressing bright topaz and cyan signals, respectively, that are co-localized with a sharp AC label and ALP staining. The AC label indicates active mineralization in the past 24 h whereas ALP staining represents ALP-positive osteoblasts. Cell nuclei are stained blue with DAPI. TRAP-positive osteoclasts are identified by their bright yellow stain that is co-localized with faint topaz/cyan cells. Host-derived fibroblasts express faint topaz and are negative for AC, ALP or TRAP. Undifferentiated donor cells are expressing faint cyan and are negative for AC, ALP or TRAP.

Referring specifically to FIG. 20A, histological evaluation revealed the capacity of CaPG to induce osteogenesis, when donor cells were abundantly present throughout the implant. The darkfield image shows the presence of white mineralized tissue throughout the implant along with strong AC labels and ALP activity. Analysis of the high magnification area at a cellular level showed co-localization of bright topaz and cyan fluorescent signals with sharp AC lines and ALP-positive osteoblasts. This implies active participation of both donor- and host-derived osteoblasts in new bone formation. The bone formation was taking place both on the outer rim and at various sites throughout the implant. Cellular infiltration was also occurring throughout the implant, as can be seen from the TB and DAPI stains, but the absence of corresponding strong fluorescent signals, indicated their non-osteoblastic phenotype. Moreover, there were areas of TRAP-positive osteoclasts within the implant demonstrating active resorption and remodeling that is taking place.

Referring specifically to FIG. 20B, when donor cyan cells were located at the implant boundaries and less distributed throughout, the implant failed to induce osteogenesis ectopically. This result was similar to the outcome when CaPG was injected alone. There was white mineralized tissue along with diffuse AC label seen within the implant, however, without being bound by theory, this could be due to the Ca ions present in the nanomaterial structure and not mineralizing osteoblasts. Despite significant cellular migration throughout the implant, the cells did not express strong fluorescent reporter signals or ALP activity that would indicate their osteogenic lineage. Nevertheless, there was active resorption taking place by TRAP-positive cells resembling osteoclasts. The differences in these responses may be due to the effects exerted by CaPG on the donor BMSCs and vice versa, and the cell non-autonomous effects of donor BMSCs. The short time period in between mixing the cells with the materials and injecting them in vivo, most likely did not allow the cells to properly anchor onto and attach to the material prior to injections, resulting in a loss of non-adhered donor cells.

Furthermore, referring specifically to FIG. 20C, there was hardly any mineralization, ALP activity or osteoblasts detected in the graphene oxide samples. There was cellular infiltration, a portion of which were fibroblasts distinguished based on their faint topaz fluorescence. No donor cyan cells were found at the implant site suggesting the superiority of CaPG to graphene oxide as a cell carrier. There was no indication of osteogenic activity in any of the graphene oxide samples (with and without cells).

The implants showed minimal to no signs of inflammation, obvious necrosis or toxicity demonstrating good biocompatibility. Both graphene oxide and CaPG implants showed cellular infiltration and material breakdown with evidence of cell uptake and clearance. In addition, histological sections from the liver, spleen and kidneys of mice injected with graphene oxide or CaPG showed no obvious tissue damage, toxicological effects or inflammation and there was no accumulation of graphene oxide or CaPG in any of these tissues.

A number of embodiments have been described. Nevertheless, it will be understood that various modifications may be made without departing from the spirit and scope of the invention. For example, some of the steps described above may be order independent, and thus can be performed in an order different from that described.

Other implementations are also within the scope of the following claims.

1. A method for functionalizing graphene oxide, comprising:
reacting graphene oxide with a phosphite compound and a metal salt in the presence of a Lewis acid to produce phosphate functionalized graphene oxide including ions of the metal.

2. The method of claim 1, wherein the metal salt comprises a metal halide salt.

3. (canceled)

4. (canceled)

5. The method of claim 1, wherein the ions of the metal comprise inducerons capable of inducing osteogenesis or osteoinductivity.

6. The method of claim 1, wherein the phosphite compound comprises an organophosphorous compound.

7. (canceled)

8. The method of claim 1, wherein the Lewis acid comprises magnesium bromide diethyl etherate.

9. The method of claim 1, wherein reacting the graphene oxide comprises:
reacting the graphene oxide with the phosphite compound in a solution containing the Lewis acid; and
adding the metal salt to the solution.

10. (canceled)

11. An apparatus comprising:
a bone scaffold construct formed of phosphate functionalized graphene oxide including metal ions.

12. The apparatus of claim 11, wherein the bone scaffold construct is formed of a powder of the phosphate functionalized graphene oxide.

13. The apparatus of claim 11, wherein the bone scaffold construct comprises a putty.

14. The apparatus of claim 11, wherein the bone scaffold construct comprises a membrane.

15. (canceled)

16. The apparatus of claim 11, wherein the bone scaffold construct has a compressive Young's modulus of between about 150 MPa and about 3 GPa.

17. (canceled)

18. The apparatus of claim 11, wherein the bone scaffold construct has an ultimate compressive strength of between about 50 MPa and about 350 MPa.

19. (canceled)

20. The apparatus of claim 11, wherein the bone scaffold construct has a compressive storage modulus between about 100 MPa and about 3 GPa.

21. (canceled)

22. (canceled)

23. (canceled)

24. The apparatus of claim 11, wherein the bone scaffold construct has a compressive loss modulus of between about 5 MPa and about 20 MPa.

25. (canceled)

26. The apparatus of claim 11, wherein the bone scaffold construct has a shear storage modulus of between about 250 MPa and about 3 GPa.

27. (canceled)

28. (canceled)

29. (canceled)

30. The apparatus of claim 11, wherein the bone scaffold construct has a shear loss modulus of between about 40 MPa and about 150 MPa.

31. (canceled)

32. (canceled)

33. The apparatus of claim 11, wherein when the bone scaffold construct is exposed to an aqueous environment for a period of up to 28 days, a compressive modulus of the bone scaffold construct changes by less than about 100%.

34. (canceled)

35. (canceled)

36. (canceled)

37. The apparatus of claim 33, wherein when the bone scaffold construct is exposed to an aqueous environment for a period of up to 28 days, the compressive modulus of the bone scaffold construct changes by less than about 10%.

38. (canceled)

39. The apparatus of claim 11, wherein the metal ions comprise inducerons capable of inducing osteogenesis or osteoinductivity.

40. The apparatus of claim 11, wherein the bone scaffold construct comprises an antimicrobial component.

41. (canceled)

42. The apparatus of claim 11, wherein the bone scaffold construct comprises mesenchymal stem cells.

43. The apparatus of claim 11, in which the phosphate functionalized graphene oxide comprises peptides covalently bound to the graphene oxide.

44. The apparatus of claim 11, in which the bone scaffold construct comprises bioactive molecules non-covalently associated to the phosphate functionalized graphene oxide.

45. The apparatus of claim 44, in which the bioactive molecules comprise bone morphogenetic protein 2.

46. A bone scaffold construct comprising:
a graphene oxide material formed in the shape of the bone scaffold construct, the graphene oxide material comprising:
graphene oxide,
phosphate moieties covalently bound to the graphene oxide, and
metal counter ions chemically associated with the phosphate moieties, the metal counter ions including one or more of calcium ions, potassium ions, lithium ions, magnesium ions, sodium ions, copper ions, manganese ions, strontium ions, vanadium ions, and zinc ions;
wherein a compressive Young's modulus of the graphene oxide material is between about 150 MPa and about 3 GPa,
wherein, when the bone scaffold construct is exposed to an aqueous environment, the graphene oxide material elutes the metal counter ions.

47. The bone scaffold construct of claim 46, comprising an antimicrobial component.

48. (canceled)

49. The bone scaffold construct of claim 46, comprising mesenchymal stem cells.

50. The bone scaffold construct of claim 46, in which the graphene oxide material comprises peptides covalently bound to the graphene oxide.

51. The bone scaffold construct of claim 46, in which the graphene oxide material comprises bioactive molecules non-covalently associated to the graphene oxide.

52. (canceled)

53. A method for forming a bone scaffold construct, comprising:
forming a powder into the bone scaffold construct, the powder comprising phosphate functionalized graphene oxide including metal ions.

54. The method of claim 53, wherein forming the powder into the bone scaffold construct comprises pressing the powder into the shape of the bone scaffold construct; and heat treating the pressed powder.

55. (canceled)

56. The method of claim 53, wherein forming the powder into the bone scaffold construct comprises using an additive manufacturing technique to form the powder into the bone scaffold construct.

57. The method of claim 53, wherein forming the powder into the bone scaffold construct comprises filtering a slurry of the powder to form a membrane.

58. (canceled)

59. (canceled)

60. (canceled)

61. (canceled)

62. (canceled)

63. (canceled)

64. A method for treating a bone defect, comprising:
administering a therapeutically effective amount of phosphate functionalized graphene oxide including metal ions.

65. (canceled)

66. (canceled)

67. (canceled)

68. (canceled)

69. The method of claim 64, wherein administering the phosphate functionalized graphene oxide comprises injecting an effective amount of a slurry of the phosphate functionalized graphene oxide into a site of the bone defect.

70. The method of claim 64, wherein administering the phosphate functionalized graphene oxide comprises surgically implanting a bone scaffold construct formed of the phosphate functionalized graphene oxide.

71. (canceled)

72. (canceled)

73. The method of claim 64, comprising inducing one or more of osteogenesis and osteoinductivity on the phosphate functionalized graphene oxide.

74. (canceled)

75. (canceled)

76. The method of claim 64, comprising eluting the metal ions from the phosphate functionalized graphene oxide.
